(19) United States
(12) Patent Application Publication
(10) Pub. No.: US 2026/0245669 A1
(43) Pub. Date: Aug. 20, 2026

(54) A COMPUTER MODEL OF THE SENSE OF SMELL

(71) Applicant: Liron Aharon Shalev, Berlin (DE)

(72) Inventor: Liron Aharon Shalev, Berlin (DE)

(21) Appl. No.: 19/489,600

(22) PCT Filed: Jul. 2, 2024
(86) PCT No.: PCT/EP2024/068581

§ 371 Date: Dec. 3, 2025

(30) Foreign Application Priority Data
Jul. 3, 2023 (DE) 10 2023 117 481.9

(51) Int. Cl. G16C 20/30; G06F 30/27; G16C 20/70

(52) U.S. Cl. CPC G16C 20/30; G06F 30/27; G16C 20/70

(57) ABSTRACT

The invention relates to computer implemented methods for the prediction of the smell of a molecule, comprising providing a first database comprising the structure, preferably the three-dimensional (3D) and/or chemical structure, of one or more olfactory receptor (OR), training a machine learning model or AI on the data comprised within the first database, providing a second database comprising (i) the (3D) structure of one or more odorant molecule and (ii) a respective textual description of the odor sensation induced by said one or more odor molecule in a human subject, training a machine learning (ML) model or artificial intelligence (AI) on the data comprised within the second database, fitting the (3D) structure of the one or more odor molecule to the (3D) structure of the one or more OR, thereby determining an affinity score for each fitted combination of the one or more odor molecule with a respective olfactory receptor, wherein the fitting comprises the use and training of a ML model or AI, wherein the affinity score is indicative for the degree of fit of a respective odor molecule to the OR, creating a comprehensive third database or third data embedding structure/layer comprising the data of the first database, the second database and the affinity score determined for each fitted combination of a respective odor molecule with a respective olfactory receptor, providing the (3D) structure of at least a first odor molecule of interest, which is not comprised within the second database, and predicting an odor sensation induced by the at least first odor molecule of interest in a human subject using machine learning or AI considering the data comprised within the third database or third data embedding structure/layer.

The invention relates to computer implemented methods for the prediction of the smell of a molecule, comprising providing a first database comprising the three-dimensional (3D) and/or chemical structure of one or more olfactory receptor (OR), training a machine learning model or AI on the data comprised within the first database, providing a second database comprising (i) the (3D) structure of one or more odorant molecule and (ii) a respective textual description of the odor sensation induced by said one or more odor molecule in a human subject, training a machine learning (ML) model or artificial intelligence (AI) on the data comprised within the second database, fitting the (3D) structure of the one or more odor molecule to the (3D) structure of the one or more OR, thereby determining an affinity score for each fitted combination of the one or more odor molecule with a respective olfactory receptor, wherein the fitting comprises the use and training of a ML model or AI, wherein the affinity score is indicative for the degree of fit of a respective odor molecule to the OR, creating a comprehensive third database or third data embedding structure/layer comprising the data of the first database, the second database and the affinity score determined for each fitted combination of a respective odor molecule with a respective olfactory receptor, providing the (3D) structure of at least a first odor molecule of interest, which is not comprised within the second database, and predicting an odor sensation induced by the at least first odor molecule of interest in a human subject using machine learning or AI considering the data comprised within the third database or third data embedding structure/layer.

### BACKGROUND OF THE INVENTION

The perception of odors is highly complex an involves after the entrance of odorants through the nose the recognition through olfactory sensory neurons (OSNs), which comprise numerous different kinds of olfactory receptors (ORs). The neural signal of the OSNs is subsequently transmitted and processed by the olfactory cortex of the brain (Fleischer et al., 2009).

Generally, the group of olfactory receptors (ORs) comprises the receptor families of odorant receptors (ORs), the vomeronasal receptors (V1Rs and V2Rs), trace amine-associated receptors (TAARs), formyl peptide receptors (FPRs), and the guanylyl cyclase GC-D, which are majorly G protein-coupled receptor proteins (GPCRs) (Fleischer et al., 2009).

At present it is noted in the art that the three dimensional structure of odorants, and optionally also their physicochemical properties, such as the boiling and vapor point and the molecular polarity, have an influence on the olfactory experience (smell) that is perceived by a subject. This correlation between the chemical structures and properties of odorants and the induced biological response is termed the Quantitative Structure Activity Relationship (QSAR) model (Dearden, 1994; Hau, and Connell, 1998).

However, in the prior art there is still a lack of precise methods and automated approaches to analyze and reliably determine the correlation between the structure of a substances and the olfactory perception it induces in a subject.

In light of the prior art there remains a significant need in the art to provide improved means for the prediction of an odor or olfactory perception of molecules.

### SUMMARY OF THE INVENTION

In light of the prior art the technical problem underlying the present invention is to provide alternative and/or improved means for the prediction of an odor or olfactory perception of molecules.

This problem is solved by the features of the independent claims. Preferred embodiments of the present invention are provided by the dependent claims.

The present invention relates in a first aspect to a computer implemented method for the prediction of the smell (odor/olfactory sensation) of a molecule, comprising the steps of:
a. Providing a first database comprising information regarding the structure, preferably the three-dimensional (3D) and/or chemical structure, of one or more olfactory receptor (OR), preferably wherein the database comprises information on the 3D structure of the ligand (or agonist) binding site of the one or more olfactory receptor,
b. Training a machine learning model or AI (receptor encoder) on the data comprised within the first database,
c. Providing a second database comprising (i) the (3D) structure of one or more odor (odorant) molecule and preferably (ii) a respective textual description of the odor sensation induced by said one or more odor molecule in a human subject,
d. Training a machine learning (ML) model or artificial intelligence (AI) (odorant encoder) on the data comprised within the second database,
d.1. Optionally providing data on the interaction of one or more olfactory receptor molecules from the first database with one or more odor molecule from the second database,
e. Fitting the (3D) structure of the one or more odor molecule to the (3D) structure of the one or more olfactory receptor (OR), preferably to the ligand binding site of the one or more OR, thereby determining an affinity score for each fitted combination of the one or more odor molecule with a respective olfactory receptor,
   wherein the fitting comprises the use and training of a ML model or AI (pairwise interactions encoder), optionally wherein the use and/or training of the ML model or AI also considers the interaction data provided in d.1.,
   wherein the (numerical value of the) affinity score is indicative for the degree of fit of a respective odor molecule to the olfactory receptor (OR), preferably to the ligand binding site of a respective OR, and is preferably indicative for the capability (and/or strength) of said respective odor molecule to (i) bind or interact with the respective olfactory receptor and/or (ii) to activate the respective olfactory receptor,
f. Creating a comprehensive third database or third data embedding structure/layer comprising the data of the first database, the second database and the affinity score determined for each fitted combination of a respective odor molecule with a respective olfactory receptor (preferably fitted in step e.),
g. Providing the (3D) structure (and/or any other structural and/or sequence information) of at least a first odor molecule of interest, which is not comprised within the second database, and
h. Predicting an odor sensation induced by the at least first odor molecule of interest in a human subject using machine learning or AI considering the data comprised within the third database or third data embedding structure/layer.

In embodiments of the present method the data comprised within the first database, the second database and the affinity scores determined for each fitted combination of a respective odor molecule with a respective olfactory receptor (obtained in step e.), and optionally further data, is stored within a third database or third data embedding structure/layer.

In embodiments, after the fitting of one or more odor molecules to one or more ORs a normalization and/or scaling of the obtained data may be performed, e.g., using a variety of methods, as a non-limiting example, t-scores may be used to normalize data across all or multiple analyzed ORs.

In embodiments, a pretraining is performed comprising the training of separate olfactory receptor and/or odor molecule encoders, preferably in unison with a pairwise interactions encoder layer (e.g., in embodiments of above steps b., d. and f).

In embodiments pre-training comprises learning latent space embeddings and/or intermolecular relationships using data from one or more of the first, second or third database or further data, such as e.g., ligand-OR interaction data and/or general ligand-receptor and/or ligand protein interaction data.

In embodiments of the method according to the invention, in step h. predicting an odor sensation induced by the at least first odor molecule of interest in a human subject is achieved by using one or more of the machine learning model(s) or AI trained in the above-mentioned steps b., d. and/or e., to predict, based on the (3D) structure (and/or any other structural and/or sequence information) of the at least first odor molecule of interest:
at least one odor molecule comprised within the third database or third data embedding structure/layer that has the highest similarity of affinity score(s) for one or more olfactory receptor(s) comprised within the third database or third data embedding structure/layer to the at least first odor molecule of interest, and/or
an odor sensation induced by the at least first odor molecule of interest in a human subject, wherein the odor sensation preferably corresponds to one or more textual descriptions of an odor sensation induced by an odor molecule in a human subject comprised within the third database or third data embedding structure/layer.

In embodiments of the method according to the invention, in step h. predicting an odor sensation induced by the at least first odor molecule of interest in a human subject comprises:
(I) Comparing the (3D) structure (and/or any other structural and/or sequence information) of the at least first odor molecule of interest and one or more of the odorant molecules comprised within the second or third database using the ML model or AI (odorant encoder) trained in d.,
(II) Fitting the (3D) structure (and/or any other structural and/or sequence information) of the at least first odor molecule of interest to the (3D) structure of one or more of the olfactory receptor (OR), preferably of the ligand binding site of said OR, using the ML model or AI (pairwise interactions encoder) trained in e., thereby determining an affinity score for each fitted combination of the at least first odor molecule of interest with the respective OR,
(III) Comparing one or more of the affinity score(s) determined in step (II) for each fitted combination of the at least first odor molecule of interest with the one or more olfactory receptor (OR), with the affinity score(s) for each combination of the one or more OR with one or more odor molecule comprised within the third database or third data embedding structure/layer,
(IV) thereby identifying at least one odor molecule comprised within the third database or third data embedding structure/layer possessing the highest similarity of affinity score(s) for the one or more OR to the at least first odor molecule of interest,

wherein the respective textual description comprised within the third database or third data embedding structure/layer of the odor sensation induced in a human subject by said one or more odor molecule(s) identified in (IV) is indicative of the odor sensation induced by the at least first odor molecule of interest in a human subject.

In embodiments finally the at least first odor molecule of interest is chemically synthesized, based on the (3D) structure provided in above step g.

In embodiments, predicting an odor sensation induced by the at least first odor molecule of interest in a human subject comprises finally generating and/or displaying (e.g., on a graphical user interface) a textual output comprising a description of the odor sensation predicted to be induced in a human subject instep h.

In embodiments the invention comprises a computer model for the prediction of the smell of a molecule, wherein the smell of a molecule refers to the (subjective) olfactory experience (odor sensation/olfactory perception) of a subject, or the (subjective) odor sensation it will generate in a subject. The description human subjects give to a smell or odor is subjective, but the present method preferably facilitates the prediction of a mean description that humans or the prediction that an expert would provide for a certain smell.

In embodiments a database, such as the first second and/or third database, comprises information on the 3D structure of the ligand binding site of one or more olfactory receptor. In embodiments a database, such as the first second and/or third database, (further) comprises information regarding/on the DNA, RNA and/or amino acid sequence of ORs and/or olfactory molecules respectively, and the training of a machine learning model or AI (receptor encoder) on the data comprised within said database(s) considers only the DNA, RNA and/or amino acid sequence data of OR and/or olfactory molecules. In embodiments also the fitting steps and/or prediction of an odor sensation only or also consider said DNA, RNA and/or amino acid sequence data. In embodiments the term ‘structural data’ may also be understood to comprise DNA, RNA and/or amino acid sequence data/information.

In preferred embodiments first database comprises structural data (the three-dimensional (3D) and/or chemical structure) of more than one olfactory receptor (OR), preferably multiple ORs. In embodiments the first database comprises structural data of preferably more than two, three, four, five, preferably of at least 10, 20, 30, 40, 50, 60, 70, 80, 90, 100, 200, 300, 400, 500, 600, 700, 1000, 1500, 2000 olfactory receptors (and/or receptor(s) involved in the perception of odorants (olfactory perception) and/or assisting (or transport) factors involved in the perception of odorants).

In preferred embodiments first database comprises (i) the (3D) structure of more than one, preferably multiple, odor (odorant) molecules and (ii) a respective textual description of the odor sensation induced by respective odor molecules in a human subject. In embodiments the second database comprises structural data of preferably more than two, three, four, five, preferably of at least 10, 20, 30, 40, 50, 60, 70, 80, 90, 100, 200, 300, 400, 500, 600, 700, 1000, 1500, 2000 odor (odorant) molecules and a respective textual description of the odor sensation induced by respective odor molecules in a human subject.

It is in embodiments advantageous if the first database and/or the second database each comprise (data on) a large number of molecules (receptors/proteins/molecules), preferably over 100 molecules, as in embodiments the present method is not simply interested in the absolute numerical value of affinities between odorant molecules and receptors and/or assistant factors, but rather is the patterns of ratios, similarities, differences and/or correlations between the affinities of odorants to different receptors and/or factors. This can be useful, for example, if an odorant molecule shows a certain ‘affinity pattern’ (binding pattern) to certain receptor structures (e.g., to related or similar structures of two or more receptors) and/or assistant factor structures (e.g., to related or similar structures of two or more factors). In embodiments a machine learning algorithm may learn from certain odorant structures and their affinity score ‘patterns’ to certain receptor or molecule structures to predict the binding or affinity pattern of a new, unknown (or just new/unknown to the model) molecule and thereby its smell or olfactory sensation/perception it induces in a subject.

In preferred embodiments the step (e.) of fitting the (3D) structure of the one or more odor molecule to the (3D) structure of the one or more olfactory receptor (OR), compresses the fitting of one or more or a variety or a multitude of odor molecules to a variety or a multitude of olfactory receptors (ORs). In preferred embodiments also an odor molecule of interest is fitted/compared to a variety or multitude of odor molecules (in step (I)) and/or subsequently fitted to a variety or a multitude of olfactory receptors (in step (II)).

In other words, in embodiments the present method aims to identify patterns arising from the determined affinities (affinity score) of odorants to different receptors and/or assistant factors. In the context of the present invention preferably not only the determined actual numerical value of the affinity score, but also the pattern emerging from the ratios between affinity scores of different receptors and/or assistant factors may be used for the different predictions described herein. A non-limiting example might be, that specific odorants must have a low-affinity score to a number of certain receptors to induce the odor perception of an orange flower, while a high affinity score to a number of certain receptors induces the perception of rose odor.

In some embodiments an affinity score comprises or constitutes multidimensional numerical values, such as vectors and/or tensors.

In some embodiments, the use of an AI or ML model for predicting the smell of a molecule has the advantage that a trained ML model or AI (e.g., comprising and/or employing one or more encoders and/or neural networks) has the capability to predict the smell of a molecule beyond the numerical affinity score determined to an OR, but also by considering further data, e.g., the (predicted) conformation (change) of a receptor induced by the interaction with an odor molecule, such that even if the AI or ML model has determined for two odor molecules the same affinity/affinity score to one or more OR (from the first database) the AI or ML model (e.g., comprising a deep neural network based encoder) is able to determine different olfactory sensations induced in a subject for said two odor molecule as, for example, both bind to the receptor with a comparable affinity but with (or inducing) a different conformation, e.g., changing general energy levels of the interaction.

Hence, in embodiments the method further comprises determining the most similar pattern (highest overlap of a pattern of affinity scores (to certain receptor/factor), between a respective odor molecule of interest and one or more odor molecules of the second or third database. Hence, in embodiments the present method determines the one or more odor molecule(s) from the second or third database that has/have the most similar receptor-binding pattern or behavior, or the most overlap in affinity scores to certain or all receptors (wherein in some embodiments the compared affinity scores do not have to be numerical identical in respect to a specific receptor, but should have the same ‘tendency’ (correlation) regarding their numerical affinity score values, wherein, depending on the embodiment and data, affinity score values regarded as having the ‘same tendency’ (correlate) may vary in their affinity scores from each other by between 0-1%, 0-5%, 0-10%, 0-15%, 0-20%, 0-30% (e.g., wherein correlating affinity scores are indicative of strong, moderate or weak binding/affinity, namely show a similar affinity pattern).

In other words, in embodiments the present method aims to find for/assign to an odor molecule of interest the odor molecules from the second or third database that have (highly) similar, comparable or identical affinities (affinity scores) to olfactory receptors/factors of the first database. In embodiments, once the odor molecules from the second or third database with the most similar affinities (affinity scores) to the odor molecule of interest have been identified the textual description of the smell of said ‘similar binding’ odor molecules from the second or third database can be used to predict the smell of the odor molecule of interest. This proceeding may in embodiments be performed by one or more ML model or AI, that was/were trained on the data of the first, second and/or third database and optional further data or data embeddings.

In some embodiments, the step of ‘providing a first database’ comprises determining (first) the three-dimensional (3D) and/or chemical structure, of one or more olfactory receptor(s) (OR), (and/or optionally of one or more receptor(s) involved in the perception of odorants (olfactory perception) and/or assisting (or transport) factors thereof/involved in the perception of odorants) from data comprising one or more nucleic acids sequences, e.g., mRNA, RNA and/or DNA sequences, and/or protein (amino acid) sequences and/or SMILES strings or other appropriate data of said one or more olfactory receptor(s) (OR) (and/or optionally of one or more receptor(s) involved in the perception of odorants (olfactory perception) and/or assisting (or transport) factors thereof/involved in the perception of odorants) thereby preferably providing/a first database comprising said structure(s). In embodiments the structure may be determined by computational means (e.g., in silico structure prediction), mathematical calculations and/or chemical methods (e.g., crystallographic methods).

In embodiments a machine learning (ML) method or model or a AI is used, which is preferably trained on the data of the first, second and/or third database or third embedding structure. In embodiments, either to save computational resources and/or as some ML models will achieve better results with a shorter (selected) list of features, only e.g., a selection or subset of the first, second and/or third database is used for the prediction and or training, e.g., comprising only the 100, 50 or 25 least (e.g., structurally) correlated receptors/factors.

In embodiments machine learning (e.g., a neural network, neural net) is used to automate the comparison of the databases, e.g., using statistical correlation. In embodiments a machine learning model or AI (e.g., a neural network or deep neural network) is trained before a prediction on the first, second and/or third database separately or in parallel, such that the AI learns the affinity (score) patterns between the odor molecule structures and the receptor (binding site) structures comprised within the databases. For example, the machine learning model/AI is trained only on molecule/receptor structures and the respective affinities between them such that, for example, the model knows how to exclude similar receptors/proteins and/or select unrelated receptors/proteins from a first or third database to reduce the number of proteins/receptors in a first or third database that are used for a prediction.

In embodiments, the textual description of a olfactory perception, such as a scent, smell or odor that a human subject would perceive (would be induced in a subject) might be expressed with terms such as e.g., “musky”, “sweetish”, “sweet”, “floral”, “fruity”, “putrid”, “tangy”, “crisp”, “sharp”, “lemon”, “citrus”, “limonene”, “musk”, “rose”, or a “rose head/top note”, “an orange middle/heart note, “a lemon base note” etc. The skilled person is aware of a multitude of scent descriptions used by perfumers and the respective field.

In embodiments, the predicted odor sensation induced by the at least first odor molecule of interest comprises an (textual) output describing the associations and their strengths of the odor sensation induced by a molecule of interest (e.g. “this new molecule reminds me of the scent 85% of limonene and 75% similarity to the smell of linalool”, or, “this reminds me with 86% similarity, the citrus aspect of the scent of heroine”.

In embodiments similarity may be expressed giving a numerical score between 0 and 10, or between 0 and 100, or a percentage between 0 and 100%, e.g., ‘to 80% similar’ or ‘a difference between affinity scores of 0.5’ etc.

In embodiments an affinity score may have a numerical value between 0.001 and 1, between 0 and 10, between 0 and 100, or between −100 and 100, between −50 and 50, between −10 and 10, or between −1 and 1, wherein the numerical value of the score is proportional (related) to the binding strength between two molecules, e.g., an odor molecule and its binding site on another molecule, e.g. an receptor or factor. Therefore, in embodiments an affinity score may have a numerical value of −100, −50, −10, −5, −3, −2, −1, −0.5, −0.05, −0.1, −0.01, 0.01, 0.05, 0.1, 0.5, 1, 2, 3, 4, 5, 6, 7, 8, 9, 10, 20, 30, 40, 50, 60, 70, 80, 90, or 100 or any numerical valued in between. In embodiments a strong/high affinity may be indicated by an affinity score of 1, while weak/low affinity is indicated with an affinity score of 0.1. In other embodiments a strong affinity may be indicated by an affinity score of 100, while weak affinity is indicated with an affinity score of 1, etc.

In embodiments ‘affinity scores’ of higher dimension may be determined, for example, vectors of numbers with length between 1-10, 10-100, 10-500, or 50-1000. Such embodiments apply, e.g., in cases where the AI or ML model is learning more complex correlations/interaction patterns between ORs and ligands (odor molecules), e.g., then ones that can be described by a single number score.

In some embodiments, wherein a machine learning approach is used to determine a binding (affinity) pattern, the machine leaning model may preferably learn/be trained how to identify specific affinity/binding patterns between a molecule structure and the structure of one or more receptors (and/or assisting factor(s)). Hence, in embodiments a machine leaning model/AI is preferably trained to or learns how to identify for an odor molecule of interest one or more odor molecule(s) from the second or third database that has/have the most similar affinity pattern or binding behavior to one or more receptor(s) (and/or assisting factor(s)) and thereby determine an odor molecule of interests odor, olfactory perception or smell. In embodiments a machine leaning model/AI is preferably trained or learns to determine the odor, olfactory perception or smell of an odor molecule of interest only based on its structure and even without determining or comparing affinity scores between odor molecules and receptors (and/or assisting factor(s)).

In embodiments in step g. (I) a specific receptor-affinity signature is determined for the odor molecule of interest and one or more odor molecules of the second or third database, and therefrom one or more odor molecule(s) of the second or third database with the highest similarity to the receptor affinity pattern of an odor molecule of interest is determined. In embodiments this is determined either statistically by calculating probabilities and/or likelihood, and/or by analyzing which receptors are always correlated with each other, e.g., a molecule with structure/domain Z binds to receptor class X and Y with a 80% likelihood.

The present method is in embodiments based on the structural information, preferably the three dimensional structural information (preferably 3D chemical structure) of receptors, e.g., GPCRs (G protein-coupled receptors), involved in the process of smelling: Including, but not exclusive to, olfactory receptors (ORs) and optionally other receptors involved in the perception/recognition of olfactory molecules (smell) and optionally also helper/transport proteins that transport an odor molecule (e.g., a small, hydrophobic, and/or volatile molecule or odorants) to a receptor, for example, OBP2A, OBP2B, etc. In embodiments the present method also considers DNA, RNA and/or amino acid sequence data of (encoding said) OR and/or olfactory molecules in addition to the structural information of the OR and/or olfactory molecules. In embodiments the present method only considers DNA, RNA and/or amino acid sequence data of (encoding said) OR and/or olfactory molecules without considering any (other) structural information.

In embodiments the term ‘olfactory receptor (OR)’ also comprises one or more (other) receptor(s) involved in the perception of odorants and/or assisting factors thereof.

In embodiments the at least one olfactory receptor is selected from the group comprising odorant receptors (ORs), formyl peptide receptors (FPRs), trace amine-associated receptors (TAARs), the vomeronasal receptors (V1Rs and V2Rs), and the guanylyl cyclase GC-D (which are majorly protein-coupled receptor proteins (GPCRs)).

In embodiments the at least one assistant factor is an odorant binding protein, such as e.g., OBP2A or OBP2B. In embodiments the at least one assistant factor is a odorant binding protein, a lipocalin or any other carrier protein for hydrophobic molecules, such as odor molecules or pheromones.

In embodiments the (3D) structure of the binding sites of all relevant receptors involved in the perception of smell may be determined. In embodiments this can be done, for example, using experimental crystallography information, computer simulation, or any machine learning model based on experimental data, genetic code, any combination of the above, or another method.

In embodiments the (3D) structure of a molecule or parts thereof, such as the agonist or ligand binding site of a receptor (e.g., of a receptor of the first database) and/or assisting factor is determined using experimental crystallography information, computer simulation, an AI and/or a machine learning (ML) model trained on experimental data, crystallography information, genetic sequence information of the respective olfactory receptor, or any combination of thereof.

In embodiments a second and/or third database comprises a curated dataset of odor molecules, which includes their chemical (3D) structures and textual odor descriptions (the descriptions are preferably provided by human experts or otherwise collected).

In embodiments a first and/or third database comprises a curated dataset of the three-dimensional (3D) (chemical) structure of one or more olfactory receptor (OR) and/or optionally of one or more receptor(s) involved in the perception of odorants (olfactory perception) and/or assisting (or transport) factors involved in the perception of odorants.

In embodiments an affinity score may be assigned for each of the odor molecules comprised within the second database indicating/ranging/grading/scoring their binding strength and/or fit to each receptor (or its binding site/pocket) comprised within a first database, or only to a subset thereof. In other embodiments, instead of determining the affinity of a first odor molecule to all receptors, using statistical and machine learning (ML) tools, the present methods identifies the dependencies and/or correlations between the chemical (3D) structure of different receptors of the first database and thereby scores/grades the affinity/binding strength and/or fit of respective odor molecules to a reduced list of receptors (e.g. setting a threshold for the correlation coefficient between different receptors, and only scoring the, for example, 10, 15, 20, 25, 30, 35, 40, 45 or 50 or 100 or between 10-500, 10-250, 10-100, 10-50 or 10-25 most uncorrelated receptors of the first database instead of all receptors comprised therein).

In embodiments, e.g., in step e., an affinity score is determined only for a subset of the odorant molecules and/or olfactory receptors comprised within the first, second and/or third databases by identifying dependencies and/or correlations between the different olfactory receptors and/or odor molecules using statistical and/or machine learning-based approaches, optionally by setting a threshold for the correlation coefficient between different receptors and/or odor molecules by assigning an affinity score for the 25-100 or 25-200 least correlated receptors and/or odor molecules.

In embodiments an affinity score can be determined using computer physics simulation—the most common method—a well-known software tool, is, for example, AutoDock Vina. At present there exist many other available software solutions using similar or different algorithms. Alternatively, an ML algorithm (For example, Gnina, “A deep learning framework for molecular docking”) or an algorithm that combines statistical and ML tools with a (computational (3D or interaction)) simulation element (e.g., algorithm, model or software) may be used.

In embodiments, e.g., in step e., an affinity score is determined using computer physics simulation, an ML algorithm, and/or an algorithm that combines statistical and ML methods with a (computational (3D or interaction)) simulation element (e.g., algorithm, model or software).

In embodiments, instead of a ‘simple’ numerical affinity score being determined more complex correlations of the interactions (e.g., affinity and/or conformational changes upon interaction) of odorant molecules and ORs can be learnt by the AI or ML model(s) and subsequently employed for predicting the smell (odor sensation) induced by an odorant molecule of interest.

In embodiments after determining respective affinity score(s), based on the existing data, the present method may predict the olfactory sensation/perception (odor) of molecules the model has preferably never seen before. In embodiments this prediction can be made using any number of statistical and machine learning tools and algorithms, including, but not exclusive to, simple statistics like comparing the affinities of unknown molecules to the ones of the known molecules in the dataset and calculating a simple distance measure. In embodiments the nearest neighbors may be selected to find the highest statistical agreement between textual odor descriptions.

In embodiments more performant methods and/or more accurate methods than basic statistics may be used, e.g., using any number of ML algorithms, for example, (deep) neural networks trained on the respective data and/or any database described herein or public databases such as PubChem or any database disclosing SMILES strings.

A database herein may be a first, second or third database as described herein, any public databases, such as PubChem or any database disclosing SMILES strings, and/or a database comprising information/data regarding the chemical compositions, chemical structures, RNA sequences, DNA sequences, peptide sequences, SMILES strings and/or a respective olfactory perception (smell/odor) of commercially available perfumes, natural products (like head space analysis of flowers, fruits, foods, beverages) and any other analytical reports describing the odorant components of a natural of artificial odorant objects or any combination thereof. In other words, in embodiments a database, such as a first, second and/or third database, comprises data regarding the structure, preferably the three-dimensional (3D) and/or chemical structure, of one or more olfactory receptor (OR) and/or odorant molecule, respectively, and optionally further data comprising the amino acid sequence and/or SMILES strings of one or more receptor and/or nucleic acid sequence, e.g., RNA and/or DNA sequence, encoding said olfactory receptor (OR) and/or odorant molecule, respectively.

In the context of the present invention, the terms ‘information’ and ‘data’ may be used interchangeably.

In embodiments the second and/or third database also comprises data on the vapor pressure and/or vapor point of the comprised odor molecules. In embodiments said data on vapor point and/or pressure may also be considered when determining the odor molecules with the highest similarity of affinity score-pattern. In embodiments the second and/or third database also comprises data on the vapor pressure, vapor point, molecular weights, partition coefficient (log P), topological polar surface area, number of hydrogen bond donors and acceptors, the number of aromatic rings and rotatable bonds, and/or other molecular properties of the comprised odor molecules. In embodiments said data on vapor point, pressure, molecular weights, partition coefficient (log P), topological polar surface area, number of hydrogen bond donors and acceptors, the number of aromatic rings and rotatable bonds, and/or other molecular properties may also be considered when determining the odor molecules with the highest similarity of affinity score-pattern.

In embodiments statistics in step g. comprise comparing the affinity score determined for the odor molecule of interest to the one or more olfactory receptor to the affinity scores of one or more odor molecule comprised within the second database, and calculating a (simple) distance measure, and/or wherein machine learning in step g. comprises determining the nearest neighbors to find the highest statistical agreement between the affinity scores of an odor molecule of the second database and the first odor molecule of interest, thereby predicting an odor sensation induced by the odor molecule of interest in a human subject, based on the textual odor descriptions of the nearest neighbor odor molecule in the second database.

In embodiments the at least first odor molecule of interest comprises a mixture of at least a first and a second odor molecule of interest,
and wherein in step h., in addition to (i) the (3D) structures of the least first and second odor molecule of interest, (ii) the relative percentage of each at least first and second odor molecule in the mixture's vapor and/or (iii) the statistical probability of each odor molecule to interact with the olfactory receptors in the nose of a human subject is provided, and wherein step i. comprises predicting an odor sensation induced in a human subject by the mixture of the at least first and second odor molecules of interest.

In some of such embodiments, the relative percentage of the at least first and second odor molecule in the mixture's vapor is determined by also considering the relative percentage of the least first and second odor molecule in the mixture and the respective vapor pressure of the mixture.

In embodiments wherein the at least one odor molecule of interest is a mixture of at least two different odor molecules a molecules relative percentage in the mixtures vapor is determined by considering the relative percentage of the different odor molecules in the mixture and/or the respective vapor pressure of the mixture.

The method comprises using the predictive model described herein above (first aspect of the invention), combined with information on the individual molecules constructing a mixture (e.g. orange essential oil, the perfume Chanel No.5, etc.).

By calculating the relative part of the molecules in the mixture and its vapor pressure, one can calculate the molecule's relative part in the mixture's vapor and the statistical probabilities of each component to interact with the olfactory/odor receptors in the nose. Active transference by OBPs (odorant-binding proteins) can but doesn't have to be used. Combining this prediction with the affinity scores for each molecule, using the same methods described above for individual molecules (1), one can predict the odor of the mixture at any given moment from its exposure until its complete drying out.

In embodiments wherein the odor (target) molecule of interest is a mixture of two or more odor (target) molecules the vapor pressure (or vapor point) of each odor molecule of interest is determined (either from data or using a mathematical or computer model). In embodiments the affinity scores of the odor molecules of interest in a mixture is determined consecutively or in parallel. In embodiments the final affinity pattern is corrected, normalized or calculated considering the difference in vapor pressure (or vapor point) between the odor molecules of interest in the mixture. For example, if the first odor molecules of interest has a lower vapor pressure (vapor point), less of said molecules would reach the nose or a subject, compared to the other molecules with a higher vapor pressure (vapor point), resulting in a reduced share in the final odor recognized by a subject.

In embodiments the second and/or third database also comprises data on the vapor pressure and/or vapor point of the comprised odor molecules. In embodiments said data on vapor point and/or pressure is also considered when determining the odor molecules with the highest similarity of affinity score-pattern. In embodiment said consideration comprises a subsequent or parallel normalization of the smell of the mixture, e.g., by weighting the impact of each odor molecule of interest in the mixture (e.g. in percentage).

In one specific non-limiting example embodiment each odor molecule Mᵢ in a mixture is represented by its embedding vector Oᵢ. This embedding vector can be obtained through either one of the previously described embodiments or any other implementation of this invention. In a step of weight determination the weights for the sum may be derived in various ways. For the sake of this example, herein the common definition in the field will be used, applying relative concentrations as the sum weights.

Subsequently, the relative concentration Cᵢ of each odor molecule Mᵢ in the air mixture is determined. This can be done through theoretical calculations or experimentally measured data such as headspace analysis using techniques such as gas chromatography (GC), mass spectrometry, or carbon-13 NMR. In a step of composite embedding of the mixture the composite embedding Oₘᵢₓ of the odor mixture are calculated as a weighted sum of the individual embeddings. The weights were defined based on the relative concentrations of the molecules.

Subsequently, the formula for the composite embedding is the sum: Oₘᵢₓ=Σwᵢ·Oᵢ where Oₘᵢₓ is the olfactory embedding vector of the mixture, wᵢ represents the relative concentration of molecule Mᵢ, and Oᵢ is the olfactory embedding vector of the molecule Mᵢ.

In embodiments of the present invention a machine learning model/algorithm or an artificial intelligence may be trained in advance of the prediction of an olfactory experience (smell) of an unknown or unassessed molecule on one or more databases, such as e.g., on a first, second and/or third database, comprising the three-dimensional (3D) structure of one or more olfactory receptor (OR), other receptors involved in smell and optionally also assisting/transport proteins (e.g., which transport odor molecules to receptors involved in the perception of a smell/odor), and/or the (3D) structure of one or more odor molecule and a respective textual description of the human odor sensation induced by said one or more odor molecule, and/or numerical data describing the affinity (e.g., affinity scores) of odor molecule to a binding site of receptors involved in the perception of an olfactory perception, such as a smell/odor (e.g., an OR).

In embodiments of the present invention a machine learning model/algorithm or an artificial intelligence may be trained in advance of the prediction of an olfactory experience (smell) of an unknown or unassessed molecule to evaluate/analyze and/or identify the three dimensional (chemical) structures of molecules (e.g., as provided in the databases herein) and to compare and/or fit a three dimensional (chemical) structure of a molecule to other three dimensional (chemical) structures of molecules, such that in embodiments an affinity score or a numerical evaluation of the fit/binding can be generated/predicted. For example, a model herein may be trained to fit a three dimensional (chemical) structure of an odor molecule, e.g., from the second database or newly predicted according to the invention, to the three dimensional binding site/pocket of a olfactory receptor or other receptors involved in olfactory perception, and/or e.g., to compare three dimensional (chemical) structures of a newly predicted molecule to the one or more of a known odor molecule, e.g., of the second database.

In embodiments the model can use one or more molecular representation(s) (e.g., the structure of an odor molecule of interest) to ‘directly’ predict a textual odor description, and/or similar odor molecules (structural neighbors), e.g., which are comprised within the second or third database, without the need to pre-calculate any affinities and/or fit any molecule/receptor structures to each others for the new predicted molecule.

The machine learning (or AI) may in embodiments use for a prediction and/or be trained on input data comprised of (‘classical’) alphabetical and numerical reproduction of chemical structures (e.g., H₂0), numerical vectors, data (e.g., XML) comprising coordinates of atoms and bonds, SMILES (Simplified Molecular Input Line Entry System) strings, InChl or InChlKey, graphs (e.g. of chemical structures comprising atoms and chemical bonds between the atoms), and/or pixel grids. In embodiments such graphs may be generated with programs such as MolView, PubChem or comparable programs/databases or e.g., as data (XML) comprising coordinates of atoms and bonds between the atoms.

In the context of the invention any machine learning-based model (AI) or algorithm is envisaged and can be applied, such as a artificial intelligence (AI), machine learning (ML) algorithms or models, neural networks (NN), deep neural networks (DNN), convolutional neural network (CNN), graph neural network (GNN), random forest-based prediction models, recurrent neural network, supervised learning, unsupervised learning, attention algorithms, and statistical diffusion models Transformers, LLMs, GPT and/or a combination thereof or anything similar. A machine-learning based model can comprise one or more layers. For example, a CNN may comprise two or more layers. A machine-learning based model my also involve one or more steps of feature selection and/or filtering. A machine-learning based model my also involve adversarial learning. In embodiments training of a machine-learning based model may comprised reinforcement learning.

In specific embodiments the present method or computer-implemented method may comprise one or more of the following steps:

Embodiments of the present method are or comprise an advanced pipeline comprising one or more computational model (e.g., ML model or AI) designed to simulate the human sense of smell using deep learning techniques. Embodiments of the present model may be used to leverage a primary dataset of olfactory receptor (OR) structures and a secondary dataset of odorant molecules to predict the olfactory profile of new molecules.

In embodiments, a model may be pre-trained using publicly available, experimental receptor-ligand interaction data and/or structural data of olfactory receptors (OR) and odorant molecules and is optionally ‘fine-tuned’ to simulate the affinity of molecules to an array (a multitude) of ORs, thereby creating a unique odor pattern.

In embodiments, the neural network architecture of the present model (e.g., ML model or AI) can be described through several key units or steps, and specific training steps or phases.

In embodiments, the method comprises initially a general protein-ligand interaction pretraining step or phase of the employed ML model or AI, which may be, for example, be performed using a first and a second database according to the present invention, optionally also a third database or embedding as disclosed herein.

In embodiments, during an initial training of a respective ML model or AI, the present method comprises training one or more specific encoders (encoder units), e.g., for receptor proteins and/or (potential) ligands, such as odorant molecules.

For example, in embodiments specific encoders, such as a receptor encoder, odorant encoder and/or pairwise interactions encoder, are generated in method steps b. Training a machine learning model or AI (receptor encoder) on the data comprised within the first database, d. Training a machine learning (ML) model or artificial intelligence (AI) (odorant encoder) on the data comprised within the second database, and/or f. Fitting the (3D) structure of the one or more odor molecule to the (3D) structure of the one or more olfactory receptor (OR), wherein the fitting comprises the use and training of a ML model or AI (pairwise interactions encoder).

In the context of artificial intelligence and machine learning approaches, an encoder or encoder units commonly (without limiting the invention thereto) converts and/or embeds input data during an initial phase of the generative process. In embodiments of the present method, such encoders preferably embed data of olfactory receptor (OR) proteins and/or odorant molecules (e.g., small molecules), preferably derived from a first, second and/or third database, into a unified latent space that models, e.g., their (3D) and/or chemical structures, physical properties, and/or chemical interactions, preferably from a first and second database and optional further data resources according to the present invention. Commonly a latent space is a (compressed) representation of the input data, wherein each dimension corresponds to a certain characteristic or feature. Such a pre-training step preferably provides an efficient method for representing both OR and odorant molecules, capturing essential interaction features to be used in subsequent step of the method.

A non-limiting example of this proceeding is disclosed in FIG. 1, which illustrates an embodiment of the architecture and pretraining of a general protein-ligand interaction model.

In embodiments, specific (or specifically trained or fine-tuned) odor receptor encoders are generated. Using, for example, pretrained receptor encoders and/or data comprised within a first database and/or second database and/or a third database or embedding, which preferably comprise detailed data of odor receptors (e.g., their chemical structure, crystal structure, ligand (agonist) binding sites, potential ligands, chemical interactions and/or relevant cell signaling pathways etc.). In embodiments, an array (multitude) of such specifically trained receptor encoders is generated and trained such that, for example one or more different or even each olfactory receptor (OR) (initially derived from the first database) is represented by a specific′ odor receptor encoder′. In embodiments, each encoder in such an array is fine-tuned to predict ligand affinities to a specific olfactory receptor (OR) with high efficiency, enhancing the model's predictive capabilities for odorant interactions.

In embodiments, a subsequently a step of ‘odor-encoder’ layer training is performed. In embodiments the constructed array of specific olfactory receptor encoders is preferably utilized along with a (comprehensive) database of odorant (and non-odorant) molecules, such as the second database or the third database or third data embedding structure/layer to train a final ‘odor-encoder’ layer. In embodiments, this layer generates unique odor embeddings for molecules by simulating their interactions with the trained olfactory receptor encoders (OR encoders), effectively mapping molecules into a high-dimensional “odor space.”

In embodiments, a training step, e.g., step b. (receptor encoder), step d. (odorant encoder) and/or e. (pairwise interactions encoder), comprises or constitutes a (pre) training and/or data collection or data embedding, which may be useful for later predictions and/or evaluations of odor molecules and their (strength/degree) of interaction with an olfactory receptor and/or their similarity to other odor molecules that are considered to induce a certain olfactory perception in a subject. In embodiments, the pretraining and/or data collection step comprises the collection or construction of a (comprehensive) dataset, database or data embedding and/or the learning of certain relationships, characteristics and dependencies etc. of various data of one or more olfactory receptors (OR) and/or odorant molecules and optionally their interactions, such as DNA sequences, crystallography data, (3D) structure data, chemical structure data, data on chemical interactions and/or signaling pathways, ligand binding sites and/or predicted structures. In an alternative embodiment, a database, dataset or embedding comprising one or more of the afore mentioned data of one or more olfactory receptors (OR) and/or odorant molecules and optionally their interactions, may be provided. In another alternative embodiment, one or more pretrained ML models or AI are provided that were trained on such databases, datasets or embeddings.

In embodiments, the pretraining and/or data collection step comprises the learning of one or more ML model(s) or AI from one or more (comprehensive) datasets or databases (e.g., a first, second and/or third database) comprising various data of one or more odor/odorant molecule and/or olfactory receptors, and optionally their interactions, such as DNA sequences, crystallography data, (3D) structure data, chemical structure data, data on chemical interactions and/or signaling pathways, ligand binding sites and/or predicted structures.

In embodiments, the pretraining and/or data collection step comprises the use of a database of (preferably experimental) crystallography (3D) information of odor molecules, olfactory receptors and/or ligand-receptor pairs/interactions (e.g., publicly available data). In embodiments, a first, second and/or third database comprise (preferably experimental) crystallography 3D information of odor molecules, olfactory receptors and/or ligand-receptor pairs/interactions (e.g., publicly available data).

In embodiments, the (3D) structure of a olfactory receptor and/or odor molecule, e.g., as disclosed in the first or second database respectively, was determined using experimental crystallography information, computer simulation, an artificial intelligence (AI) and/or a machine learning (ML) model trained on experimental data, crystallography information, genetic sequence information of the respective olfactory receptor and/or odorant molecule, or any combination of thereof.

In embodiments, the pretraining and/or data collection comprises the training of separate olfactory receptor and/or odor/odorant molecule (ligand) encoders, preferably in unison with a pairwise interactions encoder layer (e.g., in embodiments of above steps b., d. and f).

In embodiments pre-training comprises learning latent space embeddings and/or intermolecular relationships using known molecule binding (interaction) data.

In embodiments, the ‘pairwise interactions encoder’ layer/unit employs transformer-based architecture, preferably with cross-attention mechanisms to capture the complex relationships (chemical interactions) between OR atoms (e.g., at their ligand binding site) and their odor/odorant molecule ligands. In embodiments, this preferably allows all three encoding units to effectively update their representations using multi-head self-attention layers, preferably followed by feed-forward neural networks, normalization, and dropout layers.

In embodiments, a second training step/phase comprises the construction/generation of one or more specific olfactory receptor encoders. Therein, OR structures are preferably encoded using the pre-trained receptor encoding unit(s) (encoder(s)), creating a pre-set array (multitude) of embeddings. In embodiments, said pre-set array of embeddings may allow to omit the receptor encoder from subsequent training phases/steps, and/or during an inference phase/steps, such that only olfactory receptor encoders are used for final predictions, e.g., of the smell or structure of a new olfactory molecule of interest.

In embodiments an olfactory encoder training is performed. Therein, a ligand-encoder and pairwise-interactions-encoding units are ‘frozen’ (conserved, fixed or arrested), and the downstream olfactory encoder layers are preferably trained on a diverse dataset of small molecules (preferably within the size range capable of interacting with ORs) preferably by randomly masking, e.g., up to 37%, of the pairwise-encoding and predicting the olfactory-encoding generated by the full network. This conditions the network to the OR rather than a more generalized receptor. A self-distillation pipeline enhances model robustness by allowing the model to learn from its high-confidence predictions.

In embodiments, an olfactory alignment step is performed, which comprises the alignment of the (pre-) trained encoders to the second and/or third dataset or third embedding, containing odorant molecules with detailed odor descriptions. In embodiments, known odorant molecules are encoded by the (‘frozen’, arrested, conserved) pretrained odorant encoder, and paired during the alignment with each of the pre-encoded OR embeddings, and preferably subsequently analyzed by a olfactory encoding process. In embodiments, additional layers are trained to predict olfactory descriptions from olfactory embeddings.

In embodiments, the final layers of the olfactory encoder are non-frozen, resulting in rapid and efficient alignment with odor sensation induced in a human subject, e.g., a perfumer's perception. In embodiments, a final prediction of an odor sensation induced by an at least first odor molecule of interest in a human subject comprises an odorant (odor molecule) encoding step or phase. In embodiments, new odorant molecules of interest are therein represented by their structural features and embedded into a, preferably previously generated, high-dimensional vector space using the pretrained (preferably frozen) odorant molecule (ligand) encoder (‘odorant encoder’). In embodiments for the final prediction of the smell of a molecule of interest, preferably each encoder and/or model is ‘frozen’ (arrested, such that no training occurs anymore).

In embodiments, an odorant encoder consist of several independent transformer encoder layers. In embodiments, transformer encoder layers preferably process the molecular features and update their representations, preferably through multi-head self-attention mechanisms during the pretraining phase/step.

In embodiments, transformers commonly undergo self-supervised learning, preferably comprising a prior (unsupervised) pretraining step.

In embodiments, a training step/phase comprises a pairwise interaction encoder step or phase. In embodiments, representations of odorant molecules are therein combined with pre-encoded OR representations through cross-attention mechanisms. This feature (‘interaction block’) preferably uses triangular attention mechanisms to enhance the learning of cross-pair relationships, refining the molecular representations.

In embodiments, a training step/phase comprises an affinity prediction step or phase. In embodiments, the learned interaction representations are therein projected into affinity matrices through linear transformations and activation functions. In embodiments, a mixture density network (MDN) models the distribution of affinities using learned parameters for statistical potentials, aiding in ranking the molecular affinities.

In embodiments, an ‘inference’ step or phase is performed within the present method for the final prediction of an odor sensation induced by an at least first odor molecule of interest in a human subject. In embodiments, an inference step or phase comprises an odorant encoding and/or affinity simulation step or phase. In embodiments, only the odorant encoder (layers) are used to process the structural features of new molecules. In embodiments, the odorant encoder block, preferably through independent transformer layers, updates the molecular representations. In embodiments, the new odorant molecule of interest (e.g., small molecule) embedding is paired with some or all (pre-calculated) individual OR embeddings using a pairwise interactions encoder. In embodiments, this step is followed by feed-forward neural networks, normalization, and dropout layers to effectively create an odor space embedding.

In embodiments, this prediction maps the (new) odor molecule of interest into a high-dimensional “odor space” (“odor space” may be regarded e.g., as a specific embodiment of, e.g., the chemical space) where molecules are related, e.g., (exclusively) based on their simulated olfactory effects. This preferably enables the identification of (structurally diverse) odor molecules (of interest) that elicit similar olfactory responses or effects in a subject.

In embodiments, using the ‘odor space’ embedding, molecules can be analyzed and compared based on their predicted olfactory profiles using well-known (state of the art) algorithms. In embodiments, the embedding can also be forwarded to additional network layers to align the latent space with a second database, to pair perfumer descriptions with the learned olfactory embeddings.

A non-limiting example of this workflow is disclosed in FIG. 2.

In embodiments a specific training methodology may be employed comprising one or more of the following steps.

In embodiments, the final layers of the olfactory encoder are non-frozen, resulting in rapid and efficient alignment with perfumer perceptions (olfactory perception(s)).

The inventors surprisingly found that the present method provides a superior detection accuracy. In exemplary technical experiments the initial part of the system (all units up to the olfactory encoder) was validated against known benchmarks and achieved state-of-the-art levels for docking tasks. The present method demonstrated low error margins and high accuracy in predicting olfactory descriptors, with a success rate exceeding 95% on a validation dataset.

In embodiments the present method can be adapted and used for diverse set of tasks, including but not limited to: virtual screening of molecules, and all of the examples described later in this document. i.e. The Smell “Telephone”, Smell Synthesizer for VR System, Olfactory Hedonic Sensor System, Perfume Formula Reformulation, Natural Product Approximation, and an In-Silico Pipeline for the Generation and Development of Novel Fragrance and Flavor Ingredients.

In summary, the present method leverages state-of-the-art deep learning techniques and extensive pre-training to simulate the human sense of smell accurately. By encoding molecules into a high-dimensional odor space, it provides a powerful tool for fragrance design, odor detection, and other olfactory applications and can, for example, be used in any and all of the application examples detailed in this document.

In some embodiments the method for the prediction of the smell of a molecule, comprises the steps of:
a. Providing a first database comprising the structure, preferably the three-dimensional (3D) and/or chemical structure, of one or more olfactory receptor(s) (OR), and/or optionally of one or more receptor(s) involved in the perception of odorants (olfactory perception) and/or assisting (or transport) factors thereof/involved in the perception of odorants,
b. Determining the (3D) structure of the ligand binding site of the one or more olfactory receptor, and/or optionally of the one or more receptor(s) involved in the perception of odorants (olfactory perception) and/or the assisting (or transport) factors involved in the perception of odorants, comprised within the first database,
c. Providing a second database comprising (i) the (3D) structure of one or more odor molecule (odorant) and (ii) a respective textual description of the odor sensation (olfactory perception) induced by said one or more odor molecule in a (human) subject,
d. Fitting the (3D) structure of the one or more odor molecule to the (3D) structure of the ligand binding site of one or more of the (olfactory) receptor (determined in b.), and/or optionally of one or more receptor(s) involved in the perception of odorants (olfactory perception) and/or assisting (or transport) factors involved in the perception of odorants, thereby determining an affinity score for each fitted combination of the one or more odor molecule with the one or more olfactory receptor(s) and/or optionally the one or more receptor(s) involved in the perception of odorants (olfactory perception) and/or the assisting (or transport) factors involved in the perception of odorants,
   wherein a (high) affinity score indicates (is indicative of/correlates/is proportional to) a (high) degree of fit, of the one or more odor molecule to the ligand binding site of a respective (olfactory) receptor (or assisting factor), and is preferably indicative for the capability of said one or more odor molecule to bind or interact with said (olfactory) receptor (or assisting factor), and/or to activated the olfactory receptor,
e. Optionally, creating a third database comprising the data of the first database, the second database and the affinity score determined for each fitted combination of the one or more odor molecule with respective (olfactory) receptor(s) (or assisting factor).

In some embodiments the invention relates to a computer implemented method for the prediction of the smell of a molecule, comprising the steps of:
a. Providing a first database comprising the three-dimensional (3D) (chemical) structure of one or more olfactory receptor (OR) and/or optionally of one or more receptor(s) involved in the perception of odorants (olfactory perception) and/or assisting (or transport) factors involved in the perception of odorants,
b. Determining the (3D) structure of the agonist (or ligand) binding site of one or more olfactory receptor, and/or optionally of one or more receptor(s) involved in the perception of odorants (olfactory perception) and/or assisting (or transport) factors involved in the perception of odorants, comprised within the first database,
c. Providing a second database comprising (i) the (3D) structure of one or more odor molecule and (ii) a respective textual description of the odor sensation induced by said one or more odor molecule in a human subject,
d. Fitting the (3D) structure of the one or more odor molecule to the (3D) structure of the agonist (or ligand) binding site of one or more of the olfactory receptor, and/or optionally of one or more receptor(s) involved in the perception of odorants (olfactory perception) and/or assisting (or transport) factors involved in the perception of odorants, comprised within the first database, thereby determining an affinity score for each fitted combination of the one or more odor molecule with a respective (olfactory) receptor/factor,
   wherein a (high) affinity score indicates (is indicative of) a (high) degree of fit, of the one or more odor molecule to the agonist (or ligand) binding site of a respective olfactory receptor, and is preferably indicative for the capability of said one or more odor molecule to bind or interact with said (olfactory) receptor (or assisting factor), and/or to activated the olfactory receptor,
e. Optionally, creating a third database comprising the data of the first database, the second database and the affinity score determined for each fitted combination of the one or more odor molecule with respective olfactory receptor(s) (or assisting factor),
f. Providing the (3D) structure of at least one first odor molecule of interest, which is not comprised within the second database,
g. Predicting an odor sensation induced by the odor molecule of interest in a human subject comprising
   (I) fitting the (3D) structure of the at least one first odor molecule of interest to the (3D) structure of the agonist (or ligand) binding site of one or more of the olfactory receptor(s) (and/or assisting factor), for which an affinity score was determined in step d., thereby determining an affinity score for each fitted combination of the at least one first odor molecule of interest with respective (olfactory) receptor (or assisting factor),
   (II) Comparing one or more of the affinity score(s) determined for the at least one first odor molecule of interest in step g. with the affinity scores for the one or more odor molecule comprised within the third database and/or determined in step d., thereby selecting one or more odor molecule(s) from the second or third database with the most similar pattern (with the highest similarity) of affinity score(s) or affinity score pattern for one or more (olfactory) receptors (or assisting factor) determined in step g.,
      thereby predicting an odor sensation (olfactory perception/smell) induced by the odor molecule of interest in a human subject, based on one or more affinity score(s) assigned in step d. to the combination(s) of respective olfactory receptor(s) with odor molecule(s) and the textual description of the human odor sensation induced by the respective odor molecule in the second and/or third database,
      wherein the prediction comprises the use of statistics, and/or the use and/or training of a machine learning algorithm (or model).

In embodiments the computer implemented method for the prediction of the smell of a molecule, comprises the steps of:
a. Providing a first database comprising the three-dimensional (3D) (chemical) structure of one or more olfactory receptor (OR) and/or optionally of one or more receptor(s) involved in the perception of odorants (olfactory perception) and/or assisting (or transport) factors involved in the perception of odorants,
b. Determining the (3D) structure of the agonist (or ligand) binding site of one or more olfactory receptor, and/or optionally of one or more receptor(s) involved in the perception of odorants (olfactory perception) and/or assisting (or transport) factors involved in the perception of odorants, comprised within the first database,
c. Providing a second database comprising (i) the (3D) structure of one or more odor molecule and (ii) a respective textual description of the odor sensation induced by said one or more odor molecule in a human subject,
d. Fitting the (3D) structure of the one or more odor molecule to the (3D) structure of the agonist (or ligand) binding site of one or more of the olfactory receptor, and/or optionally of one or more receptor(s) involved in the perception of odorants (olfactory perception) and/or assisting (or transport) factors involved in the perception of odorants, comprised within the first database, thereby determining an affinity score for each fitted combination of the one or more odor molecule with a respective (olfactory) receptor/factor,
   wherein a (high) affinity score indicates (is indicative of) a (high) degree of fit, of the one or more odor molecule to the agonist (or ligand) binding site of a respective olfactory receptor, and is preferably indicative for the capability of said one or more odor molecule to bind or interact with said (olfactory) receptor (or assisting factor), and/or to activated the olfactory receptor,
   e. Creating a third database comprising the data of the first database, the second database and the affinity score determined for each fitted combination of the one or more odor molecule with respective olfactory receptor(s) (or assisting factor),
   f. Training a machine learning model or AI on the data comprised within the third database,
   g. Providing the (3D) structure of at least one first odor molecule of interest, which is not comprised within the second database,
   h. Predicting an odor sensation induced by the odor molecule of interest in a human subject by using the machine learning model or AI, which was trained in step f., for predicting one or more similar odor molecule (which preferably possesses a similar affinity pattern to olfactory receptors and/or assisting factors) comprised within the third database and/or an odor sensation (olfactory perception/smell) induced by the odor molecule of interest in a human subject, wherein the odor sensation (olfactory perception/smell) preferably corresponds to one or more textual descriptions of a human odor sensation comprised within the third database.

In another aspect or embodiment, the present invention relates to a computer implemented method for predicting the smell of mixtures of odor molecules.

In embodiments, e.g., in step h., in addition to (i) the (3D) structures of the least first and second odor molecule of interest, (ii) the relative percentage of each at least first and second odor molecule in the mixture's vapor and/or (iii) the statistical probability of each odor molecule to interact with the olfactory receptors in the nose of a human subject is provided, and wherein predicting an odor sensation, e.g., step i., comprises predicting an odor sensation induced in a human subject by the mixture of the at least first and second odor molecules of interest.

In embodiments of the present method may employ or comprise or constitute an olfactory hedonic sensor system. In general, a Hedonic Olfactory Model refers to a framework that categorizes odors based on their perceived pleasantness or unpleasantness, reflecting the inherent human biological response to different scents. This model leverages the natural inclinations of the human olfactory system to distinguish between odors that are generally considered “good” or “bad,” which often correspond to broader biological and evolutionary cues. The olfactory hedonic system serves a critical function in guiding human behavior by signaling the desired from the undesired. This includes distinguishing the healthy from the sick, the clean from the dirty, and the nutritious from the spoiled.

In the context of embodiments of the present invention, various versions of the models of the human sense of smell were developed by the inventors, as described herein and in the examples (e.g., NN, and classical docking models). Thereby the inventors discovered that rooting model knowledge in human biology, by using olfactory receptors, have the very desirable effect, of automatically aligning the models mathematical odor space with human olfactory hedonics. This inherent gradient can be detected in the high dimensional mathematical olfactory space, using known statistical dimensionality reduction techniques, or other clustering algorithms. That is, without the need of any additional aligning (e.g. using human based labeling, or perfumer descriptions) the models have a strong, built in tendency to separate “good” odors from “bad” ones.

In embodiments, a specialized physical sensor hedonic system may leverage the principles of a hedonic olfactory model to create advanced sensors for various industrial and medical applications. In embodiments, these sensors may be designed to detect and assess odors based on their perceived pleasantness or unpleasantness, without needing to identify the exact chemical compounds responsible for the smell. Embodiments of this approach simplify the detection process while ensuring robust and reliable odor assessment.

Possible application of embodiments of such models may be, without being limited thereto, pharmaceutical Industry (Detection of Disease), such as the early detection of diseases through breath, or body odor analysis. For example, a sensor designed to identify “unpleasant” odors in human breath that are known to be associated with various disease states. The benefits of such approach would be a non-invasive, early diagnosis, continuous monitoring.

Also food storage quality management is envisaged, such as monitoring the freshness and safety of stored food products. For example, a sensor that detects the onset of spoilage in food storage facilities by identifying unpleasant odors associated with bacterial growth or chemical degradation. The benefits of such approach may be the warranting of food safety, reduced waste, and improved inventory management.

A further application of such models may be the monitoring of the cleanliness of public toilet, such as maintaining cleanliness and hygiene in public restrooms. For example, a sensor system that continuously monitors the air in public restrooms for unpleasant odors, automatically alerting maintenance staff when cleaning is needed. The benefits of such approach would be that it enhances user experience, ensures high hygiene standards, reduces maintenance costs.

Embodiments of the method may also be applied for cell culture control, such as for monitoring the quality and condition of cell cultures in research and production environments. For example, a sensor that detects changes in the odor profile of cell cultures, indicating contamination or suboptimal growth conditions. The benefits of such approach would be that it ensures culture viability, improves research outcomes, prevents contamination.

Advantages of a hedonic sensor system according to embodiments of the present invention are the simplicity and robustness, as such a system is based on detecting general odor profiles rather than specific molecular compositions, making it simpler and more robust. For example, instead of identifying specific spoilage compounds in food, the sensor detects the overall unpleasant odor associated with spoilage.

Furthermore, such systems have broad detection capabilities, as they are capable of identifying negative states (bad smells) across various applications without needing detailed knowledge of the exact molecules involved. For example, the sensor can alert to a general bad smell in a public toilet, indicating poor cleanliness, regardless of the specific cause. Another advantage is the cost-effectiveness of such systems due to a simplified design and broad detection capabilities can lead to lower production and operational costs. For example, a single sensor type can be used across multiple applications, reducing the need for specialized equipment. in addition, real-time monitoring is possible as such a system/model provides continuous, real-time monitoring and immediate alerts, enabling prompt action to address detected issues. For example, instant alerts for restroom maintenance when unpleasant odors are detected.

Finally, a great versatility is possible, as the sensor system may be tailored for specific use cases in various industries, including pharma, food storage, public sanitation, and biotechnology. For example, customizable sensitivity settings for different environments to ensure accurate detection. In summary, a specialized physical sensor hedonic system exemplifies the practical utility of a hedonic olfactory model according to embodiments of the present invention in various industrial and medical contexts. By focusing on the detection of general odor profiles associated with pleasant or unpleasant smells, these sensors offer a simple, robust, and cost-effective solution for maintaining quality, safety, and hygiene in diverse applications. The ability to provide real-time monitoring and immediate alerts further enhances their value, making them indispensable tools in modern industry and medicine.

In a further aspect the present invention relates to a generative model for the discovery of novel olfactory or aroma molecules and mixtures thereof. Using the methods (e.g., comprising machine learning (ML) models) described herein together with any machine learning model capable of generating three dimensional structures of molecules, one can generate new, never before seen odor molecules from an odor description. Hence, in embodiments the present invention relates to computer implemented methods comprising the training of at least one a machine learning model or AI to predict a three dimensional (chemical) structure of a molecule, which would be capable of inducing a desired olfactory experience or perception (smell/odor) in a human subject, e.g., by binding to the binding site of one or more olfactory receptors or other receptors involved in odor perception. In embodiments said approach may comprise the steps of machine learning-based prediction or generation of a molecule's three dimensional (chemical) structure, the prediction of the binding (e.g., strength or fit) to the binding site of one or more olfactory receptors or other receptors involved in odor perception, and/or a subsequent prediction of an odor sensation (olfactory perception/smell) induced in a human subject by the predicted molecule, e.g., based on data regarding the odor sensation (olfactory perception) induced in a human subject by certain three dimensional (chemical) molecule structures, e.g., as disclosed/comprised in the second database.

In embodiments the invention relates to a computer implemented method for predicting the (3D) structure of an odor molecule of interest capable of inducing an odor sensation of interest in a human subject and based on its binding affinity to at least one olfactory receptor, comprising the steps of
a. Providing a first database comprising the structure, preferably the three-dimensional (3D) and/or chemical structure, of one or more olfactory receptor (OR), preferably wherein the database comprises data on the 3D structure of the ligand (or agonist) binding site of one or more olfactory receptor comprised within the first database,
b. Training a machine learning model or AI (receptor encoder) on the data comprised within the first database,
c. Providing a second database comprising (i) the (3D) structure of one or more odorant molecule and (ii) a respective textual description of the odor sensation induced by said one or more odor molecule in a human subject,
d. Training a machine learning (ML) model or artificial intelligence (AI) (odorant encoder) on the data comprised within the second database,
d.1 Optionally providing data on the interaction of one or more olfactory receptor molecules from the first database with one or more odorant molecule from the second database,
   e. Fitting the (3D) structure of the one or more odor molecule to the (3D) structure of the one or more olfactory receptor (OR), preferably to the ligand binding site of the one or more OR, thereby determining an affinity score for each fitted combination of the one or more odor molecule with a respective olfactory receptor,
      wherein the fitting comprises the use and training of a ML model or AI (pairwise interactions encoder), optionally wherein the use and/or training of the ML model or AI also considers the interaction data provided in d.1.,
      wherein the (numerical value) of the affinity score is indicative for the degree of fit of a respective odor molecule to the olfactory receptor (OR), preferably to the ligand binding site of a respective OR, and is preferably indicative for the capability of said respective odor molecule to (i) bind or interact with the respective olfactory receptor and/or (ii) to activate the respective olfactory receptor,
   f. Creating a comprehensive third database or third data embedding structure/layer comprising the data of the first database, the second database and the affinity score determined for each fitted combination of a respective odor molecule with a respective olfactory receptor,
and further comprising the steps of
   G. Selecting from the third database or third data embedding structure/layer a textual description of an odor sensation of interest induced by a first odor molecule,
   H. Determining from the third database or third data embedding structure/layer one or more olfactory receptor(s) with the highest affinity score(s) for the first odor molecule,
   I. Predicting from the data of the third database or third data embedding structure/layer the (3D) structure of an odor molecule of interest, which is not comprised within the third database,
   wherein the predicted (3D) structure is modelled/predicted to fit with a high affinity score to the (3D) structure of the ligand binding site of the one or more olfactory receptor(s) selected in H., wherein the high affinity score of the predicted (3D) structure of the odor molecule of interest is indicative for the capability of said odor molecule of interest to bind or interact with the ligand binding site of the respective one or more olfactory receptor(s), and/or
   to activated the one or more olfactory receptor(s),
   thereby inducing an odor sensation in a human subject similar to the first odor molecule.

In embodiments of said method, the prediction method used in step H. is employed by an AI and/or a machine learning (ML) model, preferably a gradient-less ML model, preferably comprising a genetic algorithm to generate molecules and/or to optimize the prediction toward a given numeric vector, or
a gradient-based ML model, preferably comprising a generative adversarial network (GAN) or a diffusion probabilistic model.

In embodiments of said method, the AI and/or a ML model is or comprises one or more of the machine learning model(s) or AI trained in b., d. and f.

In embodiments of said method, the predicted (3D) structure of the odor molecule of interest is used to chemically synthesize the predicted molecule structure.

In some other embodiments the present invention relates to a computer implemented method for the prediction of the smell of a molecule, comprising the steps of:
a. Providing a first database comprising the structure, preferably the three-dimensional (3D) and/or chemical structure, of one or more olfactory receptor(s) (OR), and/or optionally of one or more receptor(s) involved in the perception of odorants (olfactory perception) and/or assisting (or transport) factors involved in the perception of odorants,
b. Determining the (3D) structure of the agonists (or ligand) binding site of one or more olfactory receptor, and/or optionally of one or more receptor(s) involved in the perception of odorants (olfactory perception) and/or assisting (or transport) factors involved in the perception of odorants, comprised within the first database,
c. Providing a second database comprising (i) the (3D) structure of one or more odor molecule (odorant) and (ii) a respective textual description of the odor sensation (olfactory perception) induced by said one or more odor molecule in a (human) subject,
d. Fitting the (3D) structure of the one or more odor molecule to the (3D) structure of the agonist (or ligand) binding site of one or more of the (olfactory) receptor, and/or optionally of one or more receptor(s) involved in the perception of odorants (olfactory perception) and/or assisting (or transport) factors involved in the perception of odorants, thereby determining an affinity score for each fitted combination of the one or more odor molecule with a respective olfactory receptor,
   wherein the numerical value of the affinity score is preferably indicative (correlates/is proportional) with the degree of fit, of the one or more odor molecule to the agonist (or ligand) binding site of a respective olfactory receptor, and is preferably indicative for the capability of said one or more odor molecule to bind or interact with said olfactory receptor, and/or to activated the olfactory receptor,
e. Optionally, creating a third database comprising the data of the first database, the second database and the affinity score determined for each fitted combination of the one or more odor molecule with respective olfactory receptor(s),
F. Selecting from the third database an odor sensation induced by a second odor molecule of interest,
G. Determining from the third database one or more (olfactory) receptor(s) with the highest affinity scores for the second odor molecule of interest,
H. Predicting from the data of the third database the (3D) structure of a third (new) odor molecule of interest, wherein said predicted (3D) structure is modelled/predicted to fit with a (high) affinity score, preferably an affinity score that is comparable or higher than the affinity score for the second odor molecule of interest to the respective receptor, to the (3D) structure of the agonist (or ligand) binding site of the olfactory receptor selected in step G., wherein the affinity score of said predicted (3D) structure of the third odor molecule of interest is preferably indicative for the capability of said third (new) odor molecule of interest to bind or interact with said olfactory receptor, and/or to activated the olfactory receptor, and wherein the third odor molecule of interest is not comprised within the third database.

Each of the herein disclosed (above and below) features in the context of one (computer-implemented) method is considered to be disclosed I the context of any other (computer-implemented) method disclosed (above and below) herein.

In embodiments the prediction method used in step H. is a machine learning (ML) method, preferably either
i. a gradient-less ML method, preferably comprising a genetic algorithm to generate molecules and/or to optimize the prediction toward a given numeric vector, or
ii. a gradient-based ML method, preferably generative adversarial network (GAN) or a diffusion probabilistic model.

In embodiments the predicted (3D) structure of the third odor molecule of interest is used to chemically synthesize the predicted molecule structure.

For a given odor description, the reverse of the process described for the first aspect of the invention can be used to calculate/predict a list of affinities.

In embodiments the present methods may comprise the use of simple statistical tools, artificial intelligence, machine learning (ML) algorithms or models, comprising neural networks (NN), deep neural networks (DNN), convolutional neural network (CNN), graph neural network (GNN), Transformers, LLMs, GPTs, random forest-based prediction models, recurrent neural network, supervised learning, unsupervised learning, attention algorithms, and statistical diffusion models and/or a combination thereof or anything similar. A machine-learning based model can comprise one or more layers. For example a CNN may comprise two or more layers. A machine-learning based model my also involve steps of feature selection.

The new list of affinities, full or partial, or any other representation constructed using statistical or ML tools (i.e., a different embedding of the affinity and other data into a numerical vector) can then be used to predict new molecules.

The prediction can be gradient-less, e.g., using a genetic algorithm to generate molecules and optimize toward the given numeric vector. Alternatively, any other ML method may be applied, such as gradient-based or gradient-free. A non-limiting example would be GANs, diffusion probabilistic models, auto encoders etc.

Using any representation of the odorant molecules, for example, SMILES (Sharma et al., 2021), fingerprints, or 3-dimensional (3D) models like .pdb files. These molecules can then be synthesized in the organic chemistry lab and used for their odor qualities in any commercial product.

In embodiments instead or in addition to the first database public databases may be used such as the Olfactory Receptor DataBase (ORDB; Yale University, USA) and/or PubChem database.

In embodiments instead or in addition to the second database public databases may be used such as the AromaDb database (CSIR-Central Institute of Medicinal & Aromatic Plants, India), the SuperScent database (Charité, Germany) and/or PubChem database.

In another aspect the present invention relates to a apparatus, device or system. In embodiments the invention relates to a data processing (computing) apparatus, device or system comprising means for carrying out (the steps of) the method according to the invention. In embodiments the invention relates to a data processing (computing) apparatus, device or system comprising a processor adapted to/configured to perform the method according to the invention.

In another aspect the present inventio relates to a computer program comprising instructions which, when the program is executed by a computer, cause the computer to carry out (the steps of) the method according to the invention.

In another aspect the present inventio relates to a computer-readable (storage) medium or data carrier comprising instructions which, when executed by a computer, cause the computer to carry out (the steps of) the method according to the invention.

The inventors consider that embodiments of the present method may be adapted and used for a diverse set of tasks, including but not limited to virtual screening of molecules, and all of the examples described later in this document. i.e. The Smell “Telephone”, Smell Synthesizer for VR System, Olfactory Hedonic Sensor System, Perfume Formula Reformulation, Natural Product Approximation, and an In-Silico Pipeline for the Generation and Development of Novel Fragrance and Flavor Ingredients.

Embodiments of the present method may, for example, without limitation thereto be adapted and used for diverse set of tasks, including but not limited to: virtual screening of molecules, and all of the examples described later in this document. i.e. The Smell “Telephone”, Smell Synthesizer for VR System, Olfactory Hedonic Sensor System, Perfume Formula Reformulation, Natural Product Approximation, and an In-Silico Pipeline for the Generation and Development of Novel Fragrance and Flavor Ingredients.

Embodiments and features of the invention described with respect to the method for the prediction of the smell (odor sensation) and the method for predicting the (3D) structure of an odor molecule of interest capable of inducing an odor sensation of interest in a human subject are considered to be disclosed with respect to each and every other aspect of the disclosure, such that features characterizing each of the methods, may be employed to characterize the other method and vice-versa. The various aspects of the invention are unified by, benefit from, are based on and/or are linked by the common and surprising finding of predicting the smell of a molecule based on its molecular structure and vice versa, preferably using computational approaches such as ML and/or AI.

### DETAILED DESCRIPTION OF THE INVENTION

All cited documents of the patent and non-patent literature are hereby incorporated by reference in their entirety.

In the context of the invention a ‘subject’ is preferably a human subject, but may also, in embodiments be any other subject, such as a mammal, an animal or else.

In the context of embodiments of the present invention the terms ‘odor’, ‘smell’, ‘olfactory perception’, ‘olfactory sensation’, or ‘odor sensation’ or ‘odor sensation induced in a human subject’ may be used interchangeably.

In the context of embodiments of the present invention the terms ‘odor molecule’ and ‘odorant molecule’, as well as ‘smell’, ‘odor’ and ‘odorant’ may be used interchangeably.

In the context of embodiments of the present invention the terms ‘odor receptor’, ‘odorant receptor’ and ‘olfactory receptor’ may be used interchangeably.

In the context of embodiments of the present invention an ‘odor molecule’ is any odorant or molecule, e.g., a small, hydrophobic, hydrophilic and/or volatile molecule, that has the capability to induce an olfactory sensation in a subject, preferably by binding to a respective receptor, e.g., an olfactory receptor (OR), or any other receptor involved in the perception of smell.

Olfactory receptors can be derived from the database https://senselab.med.yale.edu/ORDB/.

In embodiments herein when referring to olfactory receptor(s), also other receptor(s) involved in the perception of odorants (olfactory perception) and/or assisting (or transport) factors involved in the perception of odorants may be meant as well. In other words, in embodiments the term ‘olfactory receptor (OR)’ also comprises one or more (other) receptor(s) involved in the perception of odorants (‘other’ means receptors ‘commonly’ not referred to as olfactory receptors) and/or assisting factors thereof.

Olfactory receptors (ORs), also known as odorant receptors, are chemoreceptors expressed in the cell membranes of olfactory receptor neurons and are involved in the detection of compounds with an odor (called odorants) that trigger the sense of smell. Activated olfactory receptors initiate nerve impulses that transmit information about the odor to the brain. These receptors belong to class A of the rhodopsin-like family of G protein-coupled receptors (GPCRs). Depending on their structure and location, olfactory receptors are categorized into several receptor families which comprise the odorant receptors (ORs), formyl peptide receptors (FPRs), the guanylyl cyclase GC-D, the vomeronasal receptors (V1Rs and V2Rs), and trace amine-associated receptors (TAARs).

ORs are distinguishable from other GPCRs by several conserved amino acid motifs; these include an LHTPMY motif within the first intracellular loop, the most characteristic MAYDRYVAIC motif at the end of transmembrane (TM) domain 3 (TM3), a very short SY motif at the end of TM5, an FSTCSSH stretch at the beginning of TM6 and PMLNPF in TM7 (Fleischer et al., 2009).

In embodiments herein any receptor involved in the perception of an odor, namely an receptor to which an odor molecule (odorant molecule or odorant) may bind and induce an olfactory sensation in a subject may be comprised herein under the term ‘olfactory receptor’. In embodiments herein, the first database may also comprise the structure any receptor involved in the perception of an odor, namely an receptor to which an odor molecule (odorant) may bind and induce an olfactory sensation in a subject. Herein, the terms odor molecule, odorant molecule or odorant may be used interchangeably.

In embodiments herein the first database may also comprise molecules (‘helper molecules’ or ‘assisting factors’) involved in the perception of an odor, namely molecules to which an odor molecule (odorant) may bind and that transports the odorant to a respective receptor, such that upon binding of the odorant to the receptor an olfactory sensation is induced in a subject. Such ‘helper molecules’/‘assisting factors’ may be without limitation thereto, molecules such as odorant binding proteins like OBP2A, OBP2B, LCN1 (Lipocalin 1), etc.

In general odorant-binding proteins (OBPs) are small (between 10-30 kDa) soluble proteins highly concentrated in the nasal mucus of vertebrates, such as mammals or preferably humans. Due to their affinity of OBPs toward odor molecules and pheromones OBPs are considered to play a role in olfactory perception.

A common nomenclature system has been developed for the olfactory receptor family and is the basis for the official Human Genome Project (HUGO) symbols for the genes encoding these receptors. The names of individual members of the olfactory receptor family are in the format “ORnXm”, wherein ‘OR’ is the family denotation (olfactory receptor superfamily), ‘n’ defines an integer representing a family (e.g. 1-56) whose members share more than 40% sequence identity, whereas ‘X’ stands for a letter (A, B, C, . . . ) denoting a subfamily (commonly >60% DNA sequence identity). ‘M’ represents an integer for an individual family member (isoform). An example would be OR1A1 that is the first isoform of subfamily A of olfactory receptor family 1.

Members of the same olfactory receptor subfamily (commonly >60% DNA sequence identity) are likely to recognize structurally similar odorant molecules.

Two major classes of human olfactory receptors have been identified: Class I (fish-like receptors) OR families 51-56 and Class II (tetrapod-specific receptors) OR families 1-13. Class I receptors are considered to be specialized to detect hydrophilic odorants, whereas Class II receptors detect more hydrophobic compounds.

VN1R1 is a putative human pheromone receptor that is expressed in the human olfactory mucosa.

Herein the terms olfactory experience, odor sensation or olfactory perception may preferably used interchangeably.

In the context of the present invention a odor molecule or odorants may be preferably a small, hydrophobic, and/or volatile molecule inducing a olfactory (odor/smell) sensation or perception in a subject, preferably a human subject.

In embodiments herein a subject is preferably a mammalian subject, such as an animal or human and more preferably a human subject. In embodiments the present method may also be employed for subjects other than humans, such as animals, e.g., rodents, dogs or cats.

In embodiments the term ‘fitting’, such as fitting the (3D) structure of the one or more odor molecule to the (3D) structure of the one or more olfactory receptor (OR), preferably to the ligand binding site of the one or more OR, may refer to or comprises the (computational/in silico) modelling and/or calculation of chemical parameters, physical forces and/or other interaction parameters indicative of specific (chemical/physical/atomic attraction (forces)/attractive interactions) interactions between two molecules to each other, e.g., of a ligand to a receptor, preferably to a receptors active and/or ligand binding site. In this context, the term ‘fitted combination’ may in embodiments refer to a pair/combination of a respective odor molecule with a respective olfactory receptor for which such a fitting, docking, modelling and/or respective calculations has been performed (e.g., in method step e.). In some embodiments the fitting may be performed with suitable software, such as AutoDock Vina or PyMOL etc., and/or by machine learning models or AI, e.g., based on (training on/learning from) structural and/or interaction data of certain ligands and receptor molecules (e.g., from training datasets/databases).

In general machine learning can be divided into the types of supervised learning, unsupervised learning, and reinforcement learning.

Machine learning in general refers to algorithms that use training data to construct a computational model that enables independent (majorly without external instructions/commands) predictions or decision making.

In embodiments the present method may comprise the use of simple statistical tools, artificial intelligence (AI), and/or machine learning (ML) algorithms or models, comprising preferably neural networks (NN), deep neural networks (DNN), convolutional neural network (CNN), artificial neural network (ANN), graph neural network (GNN), Transformers, LLMs, GPTs, random forest-based prediction models, recurrent neural network, supervised learning, unsupervised learning, attention algorithms, and statistical diffusion models and/or a combination thereof or anything similar. In embodiments herein the terms ‘artificial intelligence (AI)’ and ‘machine learning (ML) model’ may be used interchangeably.

Random (decision) forests are an ensemble learning method (using multiple learning algorithms for an improved predictive performance) for e.g., regression or classification tasks, builds a plurality of decision trees (supervised learning) during a training time.

Deep learning is a type of machine learning that is based on neural networks comprising multiple layers such as deep neural networks (DNN), convolutional neural networks, recurrent neural networks, deep belief networks, deep reinforcement learning, and transformers, wherein learning can be either supervise or unsupervised or semi-supervised.

An artificial neural network (ANN), described commonly a neural network that is inspired by the brains biological neural network.

Generative adversarial network (GAN) are machine learning frameworks, wherein two neural networks contest with each other in a ‘zero-sum’ game, wherein one networks gain is the other network's loss. GANs are capable of generating new data with the same statistics as the initially provided training dataset. GAN are preferably able to learn in an unsupervised manner.

Commonly convolutional neural networks (CNNs) consists of interconnected layers comprising e.g., an input layer (receiving input data), non, one or more hidden layers and an output layer (producing the final result/output). In a convolutional neural network, the hidden layers include one or more layers that perform convolutions.

A graph neural network (GNN) is an artificial neural network, which may be represented as a graph (a data structure comprising interconnected nodes and edges).

In general, embeddings or ‘embedding’ is a process employed by ML models or AI to transform ‘real-world’ data into machine ‘understandable’ or processable data. Embedding commonly refers to the process of transforming ‘real-world’ (input) data into numerical representations, wherein the input data is transformed into complex mathematical representations that reflect the relationships and inherent properties of datapoints comprised within the ‘real-world’ input data. In embodiments herein, embedding input data, e.g., derived from a first, second and/or third database into a machine readable, understandable and processable data structure that may be further used by the AI or ML model. For example, in embodiments the data generated by the method of the present invention may be, as described herein, stored in a third database or may be embedded into a numerical representation that is readable and processable by an AI or ML model, such as a third data embedding structure/layer.

Diffusion probabilistic models were developed on the basics of non-equilibrium thermodynamics. Hence, those models create a sequence of ‘diffusion steps’ (Markov chain=a stochastic model describing a sequence of possible events in which the probability of each event depends only on the state reached at the previous event) that first adds random noise data to the initial data set and afterwards learns how to denoise the data again. Diffusion models comprise commonly three major components, namely the forward and reverse process, and the sampling procedure.

Feature subset selection (FSS) is a machine learning approach that uses only a subset of the available features for machine learning. FSS is necessary in part because it is technically impossible to include all features, or because of differentiation problems when there are a large number of features but only a small number of datasets, or to avoid overfitting the model, see Bias-Variance Dilemma.

In general, feature selection is a preferred step for machine learning. It refers to the process by which a subset of relevant features (variables or predictors) are selected for use in model construction. This technique is commonly used to simplify models that are easier to interpret by researchers/users, to improve data compatibility with a learning model class, to achieve shorter training times, and to avoid drawbacks of dimensionality. Feature selection may also be useful for encoding inherent symmetries that reside in the input space. Feature Selection may also be referred to as variable selection, attribute selection, or variable subset selection. Feature selection may be used to disregard data that is irrelevant or redundant. A different method is feature extraction. Feature extraction may create new features from functions of the original features, while feature selection returns a subset of features. Feature selection techniques may be applied for a datasets comprising a plurality of features and comparatively few data or examples. A feature selection algorithm may be regarded as a combination of search technologies for new feature subsets, by applying an evaluation measure that scores different feature sets.

Generally, a dissociation constant KD and affinity are inversely related. A KD value may relate to the concentration of a molecule (the amount of molecule) needed e.g., for binding to another molecule. Hence a lower the KD value (lower concentration) indicates a higher affinity of the molecule to, e.g., the binding site of the receptor.

### FIGURES

The invention is further described by the following figures. These are not intended to limit the scope of the invention, but represent preferred embodiments of aspects of the invention provided for greater illustration of the invention described herein.

FIG. 1: Architecture and pre-training of the General Protein-Ligand Interaction Model.

FIG. 2: Embedding small molecules into a high dimensional olfactory space.

FIG. 3: Using the AutoDock Vina software to dock and score all OR x Odorant pairs.

FIG. 4: A translation model, trained using the encoder model and Dataset #2, can translate bidirectionally between Olfactory Embeddings and the type of odor descriptor provided by dataset-2. For example, translation in natural language.

FIG. 5: The process begins with a specific need of a perfumer for a fragrance molecule (1). The perfumer creates a scent description (2). The description is converted by the translation model (3) into an embedding (4). The olfactory embedding is used to guide the molecule generation algorithm (5). The generative model returns a list of molecule options along with their olfactory embeddings (6). The olfactory embeddings are translated into a human-understandable description with the help of the translation model (not shown in the diagram) and returned to the perfumer. Based on the provided descriptions, the perfumer decides (7) whether some molecules are sent for lab synthesis (8) or the description is refined and the generation process is executed again.

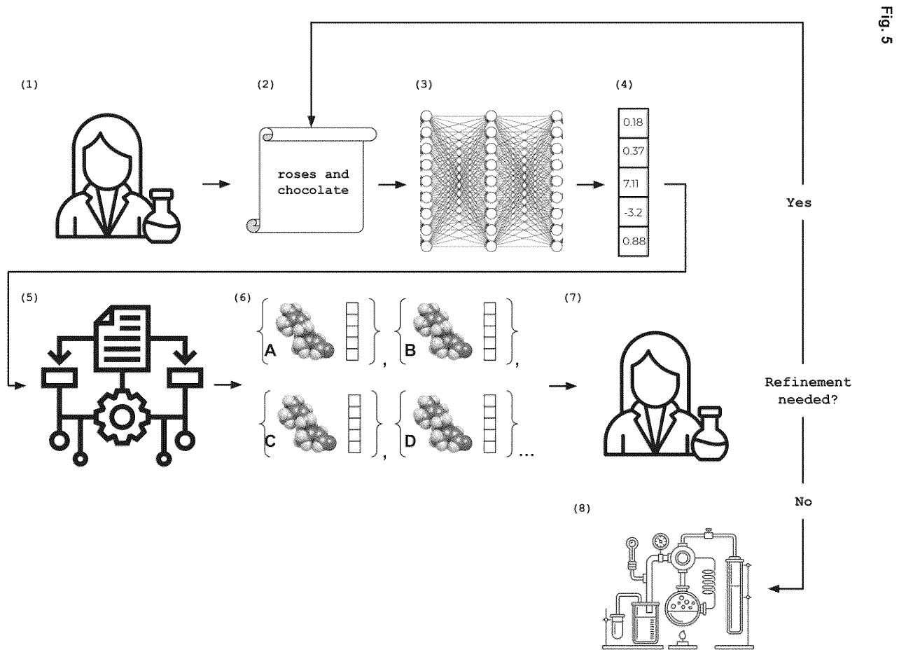

### EXAMPLES

The invention is further described by the following examples. These are not intended to limit the scope of the invention but represent preferred embodiments of aspects of the invention provided for greater illustration of the invention described herein.

Embodiments of the present method are an advanced computational model designed to simulate the human sense of smell using deep learning techniques. Embodiments of the present model leverage a primary dataset of olfactory receptor (OR) structures and a secondary dataset of odorant molecules to predict the olfactory profile of new molecules. The model is pre-trained using publicly available, experimental receptor-ligand interaction data and fine-tuned to simulate the affinity of molecules to an array of ORs, thereby creating a unique odor pattern.

### Example 1

In one embodiment, the neural network architecture of the present model was implemented as follows.

Therein a computational model (‘NN-nose’) was trained und employed in distinct phases.

### General Protein-Ligand Interaction Pretraining Phase

In the initial phase, the model primarily focused on training the protein encoder and ligand encoder units. These units embed receptor proteins and small molecules into a unified latent space that models their 3D structures, physical properties, and chemical interactions. This pretraining phase provides an efficient method for representing both receptors and ligands, capturing essential interaction features to be used in subsequent phases.

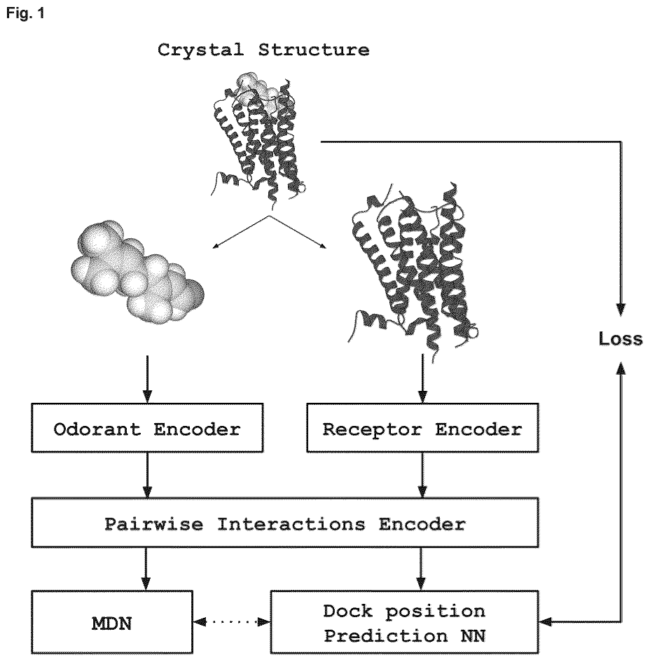

An overview of the architecture and pretraining of the General Protein-Ligand Interaction Model is shown in FIG. 1.

Specific Odor Receptor Encoder Training: Using dataset-1 (detailed dataset of odor receptors) and the pretrained encoders from the first phase, the model trained an array of specific Odor Receptor Encoders. Each encoder in this array is fine-tuned to predict ligand affinities to a specific OR with high efficiency, enhancing the model's predictive capabilities for odorant interactions.

Odor-Encoder Layer Training: The constructed array of specific OR encoders was utilized along with a comprehensive database of odorant (and non-odorant) molecules to train the final Odor-Encoder layer. This layer generates unique odor embeddings for molecules by simulating their interactions with the trained OR encoders, effectively mapping molecules into a high-dimensional “odor space.”

### Network Architecture

A subsequent training phase comprised the following steps.

### 1. Pretraining & Data Collection

A comprehensive dataset of olfactory receptor (OR) DNA sequences, crystallography data, or predicted structures was collected (referred to as dataset one).

A database of experimental crystallography 3D information of ligand-receptor pairs was used (publicly available).

Separate Receptor and Ligand Encoders were trained in unison with a Pairwise Interactions Encoder layer. These unit employed transformer-based architecture with cross-attention mechanisms to capture the complex relationships between receptor atoms and their ligands. This allows all three encoding units to effectively update their representations using multi-head self-attention layers, followed by feed-forward neural networks, normalization, and dropout layers.

### 2. Receptor Encoding

Subsequently olfactory receptor (OR) structures were encoded using the pre-trained receptor encoding unit, creating a pre-set array of embeddings that allows us to omit the protein encoder from subsequent training phases, as well as during the inference phase.

### 3. Odorant Encoding.

Odorant molecules were represented by their structural features and embedded into the same high-dimensional vector space using the pretrained Ligand Encoder.

The odorant encoder block consisted of several independent transformer encoder layers that process the molecular features and update their representations through multi-head self-attention mechanisms during the pretraining phase.

### 4. Pairwise Interactions Encoder

The representations of odorant molecules were then combined with pre-encoded olfactory receptor (OR) representations through cross-attention mechanisms.

This interaction block uses triangular attention mechanisms to enhance the learning of cross-pair relationships, refining the molecular representations.

### 5. Affinity Prediction

The learned interaction representations are projected into affinity matrices through linear transformations and activation functions.

A mixture density network (MDN) models the distribution of affinities using learned parameters for statistical potentials, aiding in ranking the molecular affinities

### Inference Phase

### 1. Odorant Encoding and Affinity Simulation;

In this example, only the odorant encoders (encoder blocks) were used to process the structural features of new molecules.

The odorant encoder block, through independent transformer layers, updated the molecular representations.

The odorant molecule (e.g., small molecule) embedding was subsequently paired with all (pre-calculated) individual olfactory receptor (OR) embeddings using the Pairwise Interactions Encoder, followed by feed-forward neural networks, normalization, and dropout layers to effectively create an Odor Space Embedding.

This pattern maps a respective molecule into a high-dimensional “odor space” where molecules are related exclusively based on their simulated olfactory effects. This enables the identification of structurally diverse molecules that elicit similar olfactory responses.

Using the odor space embedding, molecules were analyzed and compared based on their predicted olfactory profiles using well-known algorithms. In embodiments, the embedding can also be forwarded to additional network layers to align the latent space with a second database (database-2), to pair perfumer descriptions of the olfactory sensation induced in a subject with the learned olfactory embeddings.

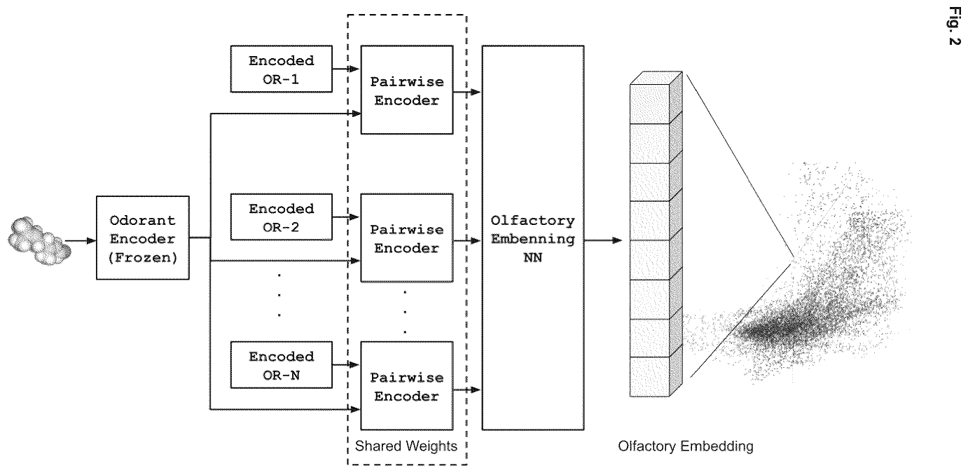

An overview of the embedding of odorant molecules (e.g., small molecules) into a high dimensional olfactory space is shown in FIG. 2.

### Training Methodology

### 1. Pre-Training

The Receptor-Encoding, Ligand-Encoding, and Pairwise-Interactions-Encoding units were pre-trained on large datasets of receptor-ligand interactions to learn general features of ligand-G protein-coupled receptor proteins (GPCR) interactions.

Pre-training preferably focuses on learning latent space embeddings and intermolecular relationships using known binding data.

### 2. Olfactory Encoder Training

The Ligand-Encoder and Pairwise-Interactions-Encoding units were ‘frozen’ (arrested; no learning), and the downstream Olfactory Encoder layers are trained on a diverse dataset of small molecules (within the size range capable of interacting with ORs) by randomly masking up to 37% of the Pairwise-Encoding and predicting the Olfactory-Encoding generated by the full network. This conditions the network to the OR rather than a more generalized receptor. A self-distillation pipeline enhances model robustness by allowing the model to learn from its high-confidence predictions.

### 3. Olfactory Alignment

The fully trained system was then aligned on dataset two, containing odorant molecules with detailed odor descriptions.

The known molecules are encoded by the frozen pretrained Odorant Encoder, paired with each of the pre-encoded OR embeddings, and processed through the Olfactory Encoding process. Additional layers are trained to predict olfactory descriptions from olfactory embeddings.

The final layers of the Olfactory Encoder are non-frozen, resulting in rapid and efficient alignment with perfumer perceptions.

### SUMMARY

The example showed a high detection accuracy of the present prediction method. The initial part of the system (all units up to the olfactory encoder) was validated against known benchmarks and achieved state-of-the-art levels for docking tasks. The present method demonstrated low error margins and high accuracy in predicting olfactory descriptors, with a success rate exceeding 95% on our validation set from the second dataset (dataset #2).

### Conclusion

The present method leveraged state-of-the-art deep learning techniques and extensive pre-training to simulate the human sense of smell accurately. By encoding molecules into a high-dimensional odor space, it provides a powerful tool for fragrance design, odor detection, and other olfactory applications and can, for example, be used in any and all of the application examples detailed in this document.

The herein employed embodiment of the present method may be adapted and used for diverse set of tasks, including but not limited to: virtual screening of molecules, and all of the examples described later in this document. i.e. The Smell “Telephone”, Smell Synthesizer for VR System, Olfactory Hedonic Sensor System, Perfume Formula Reformulation, Natural Product Approximation, and an In-Silico Pipeline for the Generation and Development of Novel Fragrance and Flavor Ingredients.

### Example 2

An embodiment of the present model utilizes classical docking methods without incorporating neural networks (NN), artificial intelligence (AI), or machine learning (ML) methodologies. Instead, it relies on well-established molecular docking techniques. Many commercial and open-source software suits offers access to this type of algorithms. For the prepose of this example we will use the freely available AutoDock Vina to build a simple model with surprisingly strong predicting power.

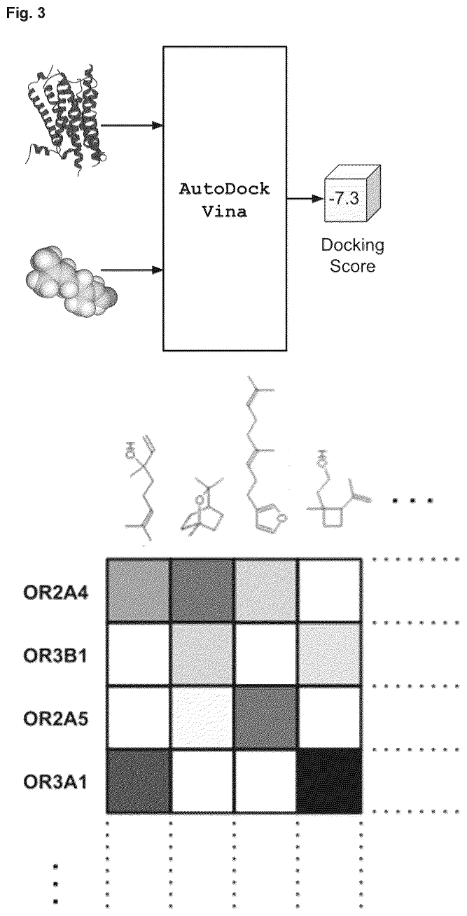

FIG. 3 shows an exemplary overview of using the AutoDock Vina software to dock and score all OR-odorant molecule pairs.

### Methodology

### Docking Phase

### 1. Data Collection Phase/Step

In a first step the present method obtains or provides a first dataset (Dataset-1), comprising the three-dimensional (3D) structures of olfactory receptors (ORs). These structures can be sourced from experimental crystallography data, computational predictions, or a combination thereof.

Subsequently, a second dataset (Dataset-2) is assembled or provided, which includes the (3D) structures of odor molecules along with textual descriptions of the odor sensations they induce in human subjects. Alternatively or in addition, any other high quality odor descriptors, produced by professional perfumers may be comprised in said second database.

### 2. Molecular Docking

First, AutoDock Vina was employed to dock each odor molecule from the second dataset (Dataset-2) to each olfactory receptor (OR) R in a first dataset (Dataset-1). Afterwards, the binding affinity scores for each molecular docking interaction are calculated. These affinity scores are indicative of the degree of fit between the odor molecule and the OR binding site. Finally, normalisation and scaling the data can be performed using a wide choice of methods. For this example, t-scores may be used to normalize across all receptors.

### 3. Affinity Score Vectors

For each molecule in the second dataset (Dataset-2), the list of it's normalized affinity scores can be seen as an embedding of the odor molecule into a high dimensional olfactory space, in which similarly smelling molecules are closer to each other.

### Odor Prediction

### Similarity Analysis

### 1. Distance Measures in Olfactory Space

Although a skilled person would expect the “curse of dimensionality” to drastically impair the ability to work with such distance measures as the L2 norm, the experimentation of the inventors have shown that thanks to biology a very stable structure are preserved within the data which allow using even these simplest of distance measures with high success.

In the present embodiments simple distance measures were therefore used to compare molecules based on their olfactory profiles. This involves calculating the Euclidean distance between the affinity score vectors of different odor molecules. Afterwards, molecules with similar odor profiles were identified by finding those with minimal distances in the olfactory space.

### 2. Odor Sensation Prediction

For a new odor molecule of interest, the molecule to all ORs in Dataset-1 were docked (fit) using AutoDock Vina and generate its affinity score vector. Afterwards, this vector was compared to the affinity score vectors of the molecules in Dataset-2 to identify the closest matches. Finally, the odor sensation induced by the new molecule based on the olfactory descriptions of the closest matching molecules in Dataset-2 was predicted.

The present embodiment (‘Relational Nose’) was tested the on the validation part of Dataset-2, revealing that this kind of model can achieve high level of accuracy (76.6%) in predicting the odor descriptions of these “unseen” odorants.

The present embodiment (‘Relational Nose’ model) may, for example, be adapted and used for diverse set of tasks, including but not limited to: virtual screening of molecules, and all of the examples described later in this document. i.e. The Smell “Telephone”, Smell Synthesizer for VR System, Olfactory Hedonic Sensor System, Perfume Formula Reformulation, Natural Product Approximation, and an In-Silico Pipeline for the Generation and Development of Novel Fragrance and Flavor Ingredients.

### Conclusion

The classical docking Relational Nose model leverages classical docking techniques to create a high-dimensional embedding of molecules in olfactory space. By evaluating the docking interactions between odor molecules and an array of ORs, the model provides a robust framework for predicting olfactory sensations and discovering new odorant molecules based on their binding profiles. This approach maintains high accuracy and reliability without the need for complex AI or ML methodologies.

### Example 3

### Embedding of Odor Mixtures

Both of the embodiments analyzed in the previous examples 1 and 2 (′NN-Nose′ and the Docking ‘Relational Nose’ models) introduce a novel concept for representing the olfactory profile of odor mixtures. This concept revolves around creating a composite pattern or embedding that captures the combined effect of multiple odor molecules. The embedding of an odor mixture is derived as a weighted sum of the individual embeddings of its constituent molecules. This approach allows for a precise and scalable representation of complex olfactory sensations.

### Methodology

### 1. Representation of Individual Molecules

First, each odor molecule Mᵢ in a mixture is represented by its embedding vector Oᵢ. This embedding vector can be obtained through either one of the previously described embodiments of examples 1 or 2 (the NN Nose, the Classical Docking Relational Nose), or any other implementation of this invention.

### 2. Weight Determination

In this step, the weights for the sum may be derived in various ways. For the sake of this example, herein the common definition in the field will be used, applying relative concentrations as the sum weights.

Subsequently, the relative concentration Cᵢ of each odor molecule Mᵢ in the air mixture is determined. This can be done through theoretical calculations or experimentally measured data such as Headspace analysis, using techniques such as gas chromatography (GC), mass spectrometry, or Carbon-13 NMR.

### 3. Composite Embedding of the Mixture

First, the composite embedding Oₘᵢₓ of the odor mixture are calculated as a weighted sum of the individual embeddings. The weights were defined based on the relative concentrations of the molecules.

Subsequently, the formula for the composite embedding is the sum:

$${O}_{mix} = \sum {w}_{i} \cdot {O}_{i}$$

where Oₘᵢₓ is the olfactory embedding vector of the mixture, wᵢ represents the relative concentration of molecule Mᵢ, and Ot is the olfactory embedding vector of the molecule Mᵢ.

### Example 4

In another example, embodiments of the present invention may be employed to capture an olfactory sensation, transmit it and then recreated at a remote location.

Such process may comprise the following steps:

### 1. Measurement of Input Odor

The odor of a sample is measured using a sensor system. A common approach is headspace gas chromatography-mass spectrometry (GC-MS), which identifies and quantifies the volatile compounds present in the air around the sample.

### 2. Encoding Detected Molecules

Each molecule detected by the sensor system is encoded using the NN Nose, the Classical Docking Nose, or any other implementation of this invention. This step involves generating an embedding vector for each molecule, which captures its olfactory properties.

### 3. Generation of Mixture Pattern

The individual embeddings of the detected molecules are combined to form a mixture pattern. This is done using the weighted sum approach described earlier:

$$E\_mix = \sum ( {C\_i}^{*} E\_i )$$

Here, C_i represents the relative concentration of molecule M_i, and E_i is the embedding vector of molecule M_i.

### 4. Transmission of Mixture Pattern

The composite embedding or mixture pattern E_mix can be stored or transmitted over a communication medium to a receiving end. This could involve sending the data over the internet, a local network, or any other suitable communication channel.

### 5. Recreation of Olfactory Sensation

At the receiving end, the system has a pre-set list of aroma ingredients that it can use to recreate the olfactory sensation. Using generative AI methods or other optimization algorithms (which can be gradient-based or gradient-free), the system generates a mixture of the available ingredients. With the goal of optimizing the resemblance of the recreated mixture to the source pattern E_mix.

### An Exemplary Workflow May be as Follows;

### 1. Measurement

A fresh orange is analyzed using headspace GC-MS. The system detects several key volatile molecules such as limonene, myrcene, and octanal.

### 2. Encoding

Each of these molecules is encoded into an embedding vector using the NN Nose model. For example: E_limonene, E_myrcene, E_octanal,

### Mixture Pattern

The relative concentrations of the molecules are determined, and the mixture pattern is calculated:

$$E\_mix = ( {C\_limonene}^{*} E\_limonene ) + ( {C\_myrcene}^{*} E\_myrcene ) + ( {C\_octanal}^{*} E\_octanal )$$

### 3. Transmission

The composite mixture pattern E_mix is transmitted to a remote location.

### 4. Recreation

At the destination, the system uses its list of available aroma ingredients. It applies a generative AI algorithm to adjust the concentrations of ingredients like synthetic limonene, myrcene, and octanal to recreate the olfactory sensation of the fresh orange.

### Conclusion

The present “telephone” example showcases the potential of digital olfactory communication. By encoding, transmitting, and recreating odor profiles, it opens up new possibilities for remote sensory experiences. Whether used for quality control in the food industry, remote diagnostics, or immersive virtual reality environments, this approach leverages advanced olfactory modeling and to bridge the gap between distant locations.

### Example 5

### Smell Synthesizer for VR System

The present example extends the capabilities of embodiments of the present invention to offer an immersive virtual reality (VR) experience by synthesizing smells based on contextual information, such as textual descriptions of a VR scene.

An exemplary workflow may be as follows:

### 1. Contextual Input

Instead of starting with a real-world odor measurement, the system receives a textual description or other forms of contextual input related to the VR scene. For example, the VR scene might describe a bustling marketplace with the aroma of fresh bread, flowers, and spices.

### 2. Pattern Generation Via Synthesizer

The synthesizer uses the external control (e.g., the textual description) to generate an olfactory pattern. This can be done using natural language processing (NLP) and generative AI models, or any other tool to generate pattern during the VR experience, or prior to it and “playing them back” during the VR session:
Textual Input: “A bustling marketplace with the aroma of fresh bread, flowers, and spices.”
NLP Processing: The system parses the textual input to identify key scent components: fresh bread, flowers, and spices.
Generative AI: The system uses a generative model to create an embedding vector for the desired olfactory experience.

### 3. Recreation of Olfactory Sensation:

The system uses its list of aroma ingredients to recreate the synthesized olfactory pattern. It applies generative AI or optimization algorithms to adjust the concentrations of the available ingredients to match the desired olfactory embedding pattern. The goal is to ensure the recreated smell mirrors the request of the VR program as closely as possible.

### Conclusion

The smell synthesizer for VR systems exemplifies how olfactory technology can enhance virtual experiences. By generating and recreating smells based on contextual inputs, such as textual descriptions, the system significantly elevates the immersive quality of VR environments. This capability can be utilized in a variety of applications, including gaming, virtual tourism, training simulations, and therapeutic environments, making the virtual experience more realistic and engaging.

### Example 6

### Perfume Formula Reformulation

Reformulating a perfume formula to replace an ingredient without significantly altering the perceived fragrance is a complex task traditionally performed by experienced perfumers. However, this process can be automated using embodiments of the models according to the present invention and additional computational methods. An exemplary workflow may be as follows:

### 1. Generate a Pattern of the Original Formula Mixture:

Similar to the “telephone” example, the first step involves generating an olfactory pattern for the original perfume formula. This pattern represents the collective scent profile created by the ingredients and their respective ratios.

Example: Suppose the original perfume formula includes ingredients A, B, C, D, and E in specific ratios. An olfactory pattern O_original is generated that captures the combined scent profile of these ingredients. An olfactory embedding pattern for the mixture is acquired, using either experimental methods (e.g. a head space analysis of the current perfume mixture) or calculating a target olfactory pattern, for example by using a weighted sum of the ingredients patterns, where the weight is proportional to their concentration in the formula as well as their relative vapor pressures.

### 2. Remove the Unwanted Ingredient:

Identify and remove the ingredient that needs to be replaced. This might be due to reasons such as availability, cost, or regulatory changes.

Example: Ingredient D needs to be removed. The resultant formula is now composed of ingredients A, B, C, and E.

The reformulation process can also accommodate additional constraints, such as using only natural ingredients, ensuring stability in specific conditions (e.g., strong basic cleaners), or staying within a certain cost range. Example: Suppose the reformulated perfume must only contain natural ingredients and be below a specific price. The generative model takes these constraints into account while optimizing the new formula.

### 3. Approximate the Original Pattern Using Minimal Additions and Modifications:

The goal is to approximate the original olfactory pattern O_original as closely as possible by adjusting the remaining ingredients and potentially adding new ones. Use generative AI models or optimization algorithms to find the optimal combination and ratios of the remaining and new ingredients to match O_original.

Example: Introduce potential replacements for D (let's say F and G) and adjust the ratios of A, B, C, E, F, and G to form a new pattern O_new that approximates O_original.

An exemplary workflow may be as follows:

### 1. Generate a Pattern of the Original Formula Mixture:

Original formula: Ingredients A, B, C, D, E
Original ratios: 20%, 25%, 15%, 30%, 10%
O_original: Olfactory pattern generated based on the above ingredients and ratios.

### 2. Remove the Unwanted Ingredient:

Unwanted Ingredient: D
Remaining formula: Ingredients A, B, C, E

### 3. Approximate the Original Pattern:

Potential replacements: F, G
Optimization process: Adjust the ratios of A, B, C, E and introduce F and G to create O_new.
Objective: Ensure O_new is as close as possible to O_original.
New formula: Ingredients A, B, C, E, F, G with optimized ratios.

### Conclusion

The perfume formula reformulation process can be significantly streamlined using AI and computational methods. By generating an olfactory pattern of the original formula, removing the unwanted ingredient, and optimizing the remaining formula to approximate the original scent, the process becomes more efficient and less reliant on manual labor. Additionally, constraints such as ingredient type, stability requirements, and cost can be seamlessly integrated into the optimization process, ensuring that the new formula meets all necessary conditions while maintaining the desired fragrance profile.

### Example 7

### Natural Product Approximation

Approximating the scent profile of a natural product (e.g., a flower, fruit, or natural environment) using a combination of perfumery ingredients is a task that can be approached similarly to reformulating a perfume. However, in this case, the original pattern is derived from a natural source rather than an existing perfume formula.

An exemplary workflow of solving this task using embodiment of the present invention may be as follows:

### 1. Generate a Pattern of the Natural Source

The first step involves capturing the olfactory pattern of the natural product. This can be done using techniques such as gas chromatography-mass spectrometry (GC-MS) to analyze the volatile compounds emitted by the natural source.

Example: Suppose the natural product is a rose flower. An olfactory pattern O_natural is generated that represents the scent profile of the rose.

### 2. Approximate the Natural Pattern Using Perfumery Ingredients

The goal is to approximate the natural olfactory pattern O_natural as closely as possible using available perfumery ingredients. Use generative AI models or optimization algorithms to find the optimal combination and ratios of perfumery ingredients that match O_natural.

Example: Use synthetic or natural ingredients like geranium oil, citronellol, nerol, and phenylethyl alcohol to form a new pattern O_approx that approximates O_natural.

### 3. Apply Additional Restrictions if Necessary:

The approximation process can also accommodate additional constraints, such as using only natural ingredients, ensuring stability in specific conditions (e.g., temperature, PH level), or staying within a certain cost range.

Example: Suppose the approximated product must only contain natural ingredients and be stable in laundry detergent. The generative model takes these constraints into account while optimizing the new formula.

### Conclusion

Approximating the scent profile of a natural product using perfumery ingredients involves capturing the olfactory pattern of the natural source, identifying key compounds, and optimizing a combination of perfumery ingredients to match the natural scent. By using generative AI models or optimization algorithms, this process can be made efficient and can incorporate additional constraints such as ingredient type, stability requirements, and cost. The result is a formula that closely approximates the natural scent while meeting all specified conditions.

### Example 8

In embodiments the present method may be employed in the context of an in silico pipeline for the generation and development of novel fragrance and flavor ingredients

### Overview

The utility of the in silico pipeline for the generation and development of novel fragrance and flavor ingredients is built upon any of the described models of the human olfactory system. This model and the corresponding development pipeline offer a transformative approach to the traditionally complex and costly process of creating new scent and flavor molecules. By leveraging the above described models and datasets, this system streamlines the process, making it more efficient, cost-effective, and environmentally friendly.

An exemplary workflow of using an embodiment of the present invention may be as follows:

### 1. Database #2

A database of molecules together with olfactory descriptions created by a perfumer.

### 2. Encoder Model

Any variation of the described model of the human sense of smell. Including but not exclusive to the NN-Node and Classical Docking Nose described above

### 3. Translation Model

Function: Converts olfactory encodings into human-understandable scent descriptions.
Mechanism: Utilizes a multimodal deep neural network to embed both olfactory encodings and human-language scent descriptions in the same latent space.

### 4. Generative Model

Function: Produces new molecular structures that conform to desired olfactory and other properties.
Mechanism: Guided by the encoder model's olfactory encodings and additional criteria like molecule size, atom types, synthesizability, and production costs.

### 5. Iterative Evaluation and Refinement

Process: Select molecules generated by the system can be synthesized in a laboratory, evaluated by perfumers, and fed back into the system for continuous improvement.

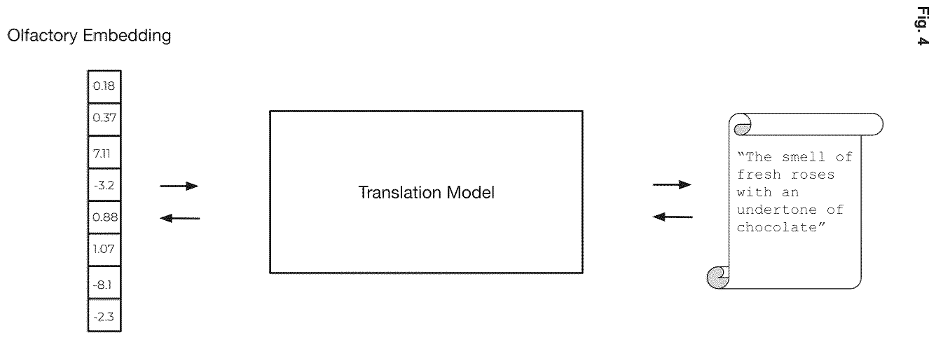

An example of such an translation model, trained using the encoder model and Dataset #2, which can translate bidirectionally between Olfactory Embeddings and the type of odor descriptor provided by dataset-2, for example, translation in natural language is shown in FIG. 4.

A more detailed workflow may be as follows:

### 1. Initial Scent Description:

The process begins with a perfumer providing a detailed description of the desired scent. Either in natural language (e.g., “a fresh, floral aroma with a hint of citrus”). Or any other structured way of describing an odor. Including perfumery notes, odor types, association to other ingredients of chemicals. Positive “The odor of Vanillin”, or negative (‘An odor to counter, mask and eliminate the malodor of stale cigarette smoke’).

### 2. Translation to Olfactory Embedding:

The translation model converts these olfactory descriptors/description into an olfactory embedding, a numeric vector, suitable for guiding the generative model.

### 3. Molecule Generation:

The generative model uses the olfactory embedding to produce candidate molecules. These molecules are not only evaluated for their scent profile, other properties (e.g. molecule size, synthesizability, cost-effectiveness, etc.) can be used as additional signals for the generative algorithm.

### 4. Perfumer Review:

The perfumer reviews the proposed molecules and their corresponding scent descriptions, as generated by the translation model. Suitable candidates are selected for synthesis.

### 5. Synthesis and Evaluation:

Selected molecules are synthesized in a chemical laboratory and then evaluated by the perfumer for their olfactory qualities, stability, and compatibility with other ingredients.

### 6. Feedback Loop:

Insights from the evaluations can be used to refine the models. This iterative process ensures continuous improvement in the accuracy and reliability of the system.

Advantages of the above-described approach are an increased efficiency, as the embodiment drastically reduces the number of laboratory experiments needed to identify viable scent molecules, saving time and resources. Further, a cost-effectiveness is achieved, as the method lowers research and development costs by minimizing the need for extensive chemical synthesis and testing. Moreover, an advantageous environmental impact is achieved, as the method reduces the consumption of chemical reagents, thereby lessening the ecological footprint of fragrance and flavor development. Also, the precision is improved, as the present method offers high control over the generated molecules, ensuring they meet specific olfactory profiles and other desired properties. Finally, the present approach enables the creation of novel fragrance and flavor molecules that might not be discovered through traditional methods.

Such methods may be employed in the area of the fragrance industry for the creation of new perfume ingredients with unique scent profiles. For example, for developing a new floral scent that is both attractive and environmentally friendly. Also, such methods may be employed for designing new flavor molecules for food and beverages. For example, for creating a cost effective alternatives for expensive spices like cardamom. The method may further be employed in the context of regulatory compliance for developing replacements for banned or restricted fragrance molecules. For example for generating alternatives to molecules like Lyral and Lilial, which have been restricted due to environmental and health concerns.

### Conclusion

The embodiments employing an in silico pipeline for the generation and development of novel fragrance and flavor ingredients represents a groundbreaking advancement in the field. By harnessing the power of artificial intelligence and a deep understanding of the human olfactory system, this approach promises to revolutionize the creation of new scent and flavor molecules. It offers significant benefits in terms of efficiency, cost, environmental impact, and innovation, making it an invaluable tool for the fragrance and flavor industries.

### REFERENCES

Fleischer J, Breer H, Strotmann J. Mammalian olfactory receptors. Front Cell Neurosci. 2009 Aug. 27; 3:9. doi: 10.3389/neuro.03.009.2009. PMID: 19753143; PMCID: PMC2742912.
John C. Dearden, Quantitative structure-activity relationships (QSAR) and odour, Food Quality and Preference, Volume 5, Issues 1-2, 1994, Pages 81-86.
Hau, K. M. and Connell, D. W. (1998), Quantitative Structure-Activity Relationships (QSARs) for Odor Thresholds of Volatile Organic Compounds (VOCs). Indoor Air, 8:23-33. https://doi.org/10.1111/j.1600-0668.1998.101-3-00004.x

1. A computer implemented method for the prediction of the smell of a molecule, comprising the steps of:
a. Providing a first database comprising information regarding the structure, preferably the three-dimensional (3D) and/or chemical structure, of one or more olfactory receptor (OR),
b. Training a machine learning model and/or AI (receptor encoder) on the data comprised within the first database,
c. Providing a second database comprising (i) the (3D) structure of one or more odor molecule and (ii) a respective textual description of the odor sensation induced by said one or more odor molecule in a human subject,
d. Training a machine learning (ML) model and/or artificial intelligence (AI) (odorant encoder) on the data comprised within the second database,
e. Fitting the (3D) structure of the one or more odor molecule to the (3D) structure of the one or more olfactory receptor (OR), preferably to the ligand binding site of the one or more OR, thereby determining an affinity score for each fitted combination of the one or more odor molecule with a respective olfactory receptor,
wherein the fitting comprises the use and training of a ML model and/or AI (pairwise interactions encoder),
wherein the (numerical value of the) affinity score is indicative for the degree of fit of a respective odor molecule to the olfactory receptor (OR), preferably to the ligand binding site of a respective OR, and is preferably indicative for the capability of said respective odor molecule to (i) bind and/or interact with the respective olfactory receptor and/or (ii) to activate the respective olfactory receptor,
f. Creating a comprehensive third database and/or third data embedding structure comprising the data of the first database, the second database and the affinity score determined for each fitted combination of a respective odor molecule with a respective olfactory receptor,
g. Providing the (3D) structure of at least a first odor molecule of interest, which is not comprised within the second database, and
h. Predicting an odor sensation induced by the at least first odor molecule of interest in a human subject using machine learning and/or AI considering the data comprised within the third database and/or third data embedding structure.

2. The computer implemented method according to claim 1, wherein h. predicting an odor sensation induced by the at least first odor molecule of interest in a human subject is achieved by using one or more of the machine learning model(s) and/or AI trained in steps b., d. and e. of claim 1, to predict, based on the (3D) structure of the at least first odor molecule of interest:
at least one odor molecule comprised within the third database and/or third data embedding structure that has the highest similarity of affinity score(s) for one or more olfactory receptor(s) comprised within the third database and/or third data embedding structure to the at least first odor molecule of interest, and/or
an odor sensation induced by the at least first odor molecule of interest in a human subject, wherein the odor sensation preferably corresponds to one or more textual descriptions of an odor sensation induced by an odor molecule in a human subject comprised within the third database and/or third data embedding structure.

3. The computer implemented method according to claim 1, wherein step h. predicting an odor sensation induced by the at least first odor molecule of interest in a human subject comprises:
(I) Comparing the (3D) structure of the at least first odor molecule of interest and one or more of the odorant molecules comprised within the second and/or third database using the ML model and/or AI (odorant encoder) trained in d.,
(II) Fitting the (3D) structure of the at least first odor molecule of interest to the (3D) structure of one or more of the olfactory receptor (OR), preferably of the ligand binding site of said OR, using the ML model and/or AI (pairwise interactions encoder) trained in e., thereby determining an affinity score for each fitted combination of the at least first odor molecule of interest with the respective OR,
(III) Comparing one or more of the affinity score(s) determined in step (II) for each fitted combination of the at least first odor molecule of interest with the one or more olfactory receptor (OR), with the affinity score(s) for each combination of the one or more OR with one or more odor molecule comprised within the third database and/or third data embedding structure,
(IV) thereby identifying at least one odor molecule comprised within the third database and/or third data embedding structure possessing the highest similarity of affinity score(s) for the one or more OR to the at least first odor molecule of interest,
wherein the respective textual description comprised within the third database and/or third data embedding structure/layer of the odor sensation induced in a human subject by said one or more odor molecule(s) identified in (IV) is indicative of the odor sensation induced by the at least first odor molecule of interest in a human subject.

4. The method according to claim 1, wherein finally the at least first odor molecule of interest is chemically synthesized, based on the (3D) structure provided in step g. of claim 1.

5. The method according to claim 1, wherein the at least one olfactory receptor is selected from the group comprising olfactory receptors (ORs), formyl peptide receptors (FPRs), the guanylyl cyclase GC-D, the vomeronasal receptors (V1 Rs and V2Rs), and trace amine-associated receptors (TAARs).

6. The method according to claim 1, wherein the (3D) structure of an olfactory receptor and/or odor molecule was determined using experimental crystallography information, computer simulation, an artificial intelligence (AI) and/or a machine learning (ML) model trained on experimental data, crystallography information, genetic sequence information of the respective olfactory receptor and/or odor molecule, or any combination of thereof.

7. The method according to claim 1, wherein in step e. an affinity score is determined only for a subset of the odor molecules and/or olfactory receptors comprised within the first, second and/or third database by identifying dependencies and/or correlations between the different olfactory receptors and/or odor molecules using statistical and/or machine learning-based approaches, optionally by setting a threshold for the correlation coefficient between different receptors and/or odor molecules by assigning an affinity score for the 25-100 least correlated receptors and/or odor molecules.

8. The method according to claim 1, wherein in step e. an affinity score is determined using computer physics simulation, an ML algorithm, and/or an algorithm that combines statistical and ML methods with a (computational) simulation element.

9. The method according to claim 1,
wherein the at least first odor molecule of interest comprises a mixture of at least a first and a second odor molecule of interest,
and wherein in step h., in addition to (i) the (3D) structures of at least the first and second odor molecule of interest, (ii) the relative percentage of each at least first and second odor molecule in the mixture's vapor and/or (iii) the statistical probability of each odor molecule to interact with the olfactory receptors in the nose of a human subject is provided, and wherein step h. comprises predicting an odor sensation induced in a human subject by the mixture of the at least first and second odor molecules of interest.

10. The method according to claim 9, wherein the relative percentage of the at least first and second odor molecule in the mixture's vapor is determined by further considering the relative percentage of at least the first and second odor molecule in the mixture and/or the respective vapor pressure of the mixture.

11. A computer implemented method for predicting the (3D) structure of an odor molecule of interest capable of inducing an odor sensation of interest in a human subject and based on its binding affinity to at least one olfactory receptor, comprising the steps of:
a. Providing a first database comprising information regarding the structure, preferably the three-dimensional (3D) and/or chemical structure, of one or more olfactory receptor (OR),
b. Training a machine learning model and/or AI (receptor encoder) on the data comprised within the first database,
c. Providing a second database comprising (i) the (3D) structure of one or more odor molecule and (ii) a respective textual description of the odor sensation induced by said one or more odor molecule in a human subject,
d. Training a machine learning (ML) model and/or artificial intelligence (AI) (odorant encoder) on the data comprised within the second database,
e. Fitting the (3D) structure of the one or more odor molecule to the (3D) structure of the one or more olfactory receptor (OR), preferably to the ligand binding site of the one or more OR, thereby determining an affinity score for each fitted combination of the one or more odor molecule with a respective olfactory receptor,
wherein the fitting comprises the use and training of a ML model and/or AI (pairwise interactions encoder),
wherein the (numerical value of the) affinity score is indicative for the degree of fit of a respective odor molecule to the olfactory receptor (OR), preferably to the ligand binding site of a respective OR, and is preferably indicative for the capability of said respective odor molecule to (i) bind and/or interact with the respective olfactory receptor and/or (ii) to activate the respective olfactory receptor,
f. Creating a comprehensive third database and/or third data embedding structure comprising the data of the first database, the second database and the affinity score determined for each fitted combination of a respective odor molecule with a respective olfactory receptor,
g. Selecting from the third database and/or third data embedding structure a textual description of an odor sensation of interest induced by a first odor molecule,
h. Determining from the third database and/or third data embedding structure one or more olfactory receptor(s) with the highest affinity score(s) for the first odor molecule,
i. Predicting from the data of the third database and/or third data embedding structure the (3D) structure of an odor molecule of interest, which is not comprised within the third database,
wherein the predicted (3D) structure is modelled/predicted to fit with a high affinity score to the (3D) structure of the ligand binding site of the one or more olfactory receptor(s) selected in h., wherein the high affinity score of the predicted (3D) structure of the odor molecule of interest is indicative for the capability of said odor molecule of interest to bind and/or interact with the ligand binding site of the respective one or more olfactory receptor(s), and/or
to activated the one or more olfactory receptor(s),
thereby inducing an odor sensation in a human subject similar to the first odor molecule.

12. The method according to claim 11, wherein the prediction method used in step h. is employed by an AI and/or a machine learning (ML) model, preferably a gradient-less ML model, preferably comprising a genetic algorithm to generate molecules and/or to optimize the prediction toward a given numeric vector, and/or
a gradient-based ML model, preferably comprising a generative adversarial network (GAN) and/or a diffusion probabilistic model.

13. The method according to claim 12, wherein the AI and/or a ML model is or comprises one or more of the machine learning model(s) and/or AI trained in b., d. and e.

14. The method according to claim 11, wherein the predicted (3D) structure of the odor molecule of interest is used to chemically synthesize the predicted molecule structure.
